(19) United States
(12) Patent Application Publication
(10) Pub. No.: US 2014/0171834 A1
(43) Pub. Date: Jun. 19, 2014

(54) Electronic-Movement Analysis Tool for Motor Control Rehabilitation and Method of Using the Same

(71) Applicant: ELIZABETHTOWN COLLEGE, Elizabethtown, PA (US)

(72) Inventors: Kurt M. DeGoede, Mount Joy, PA (US); Daniel Panchik, Hershey, PA (US)

(73) Assignee: ELIZABETHTOWN COLLEGE, Elizabethtown, PA (US)

(21) Appl. No.: 14/058,922

(22) Filed: Oct. 21, 2013

Related U.S. Application Data
(60) Provisional application No. 61/716,528, filed on Oct. 20, 2012.

(51) Int. Cl. A61B 5/11; A61B 5/00

(52) U.S. Cl. CPC A61B 5/1126; A61B 5/1101; A61B 5/742; A61B 5/1124; A61B 5/6802
USPC 600/595

(57) ABSTRACT

The present invention relates to a low cost motion capture system for clinical utilizations, such as for upper-extremity (UE) motor control rehabilitation. As contemplated herein, the present invention records range of motion data and graphs rotation, flexion, and abduction motions of a targeted joint in real time. In certain embodiments, the hardware of the system may include a simple motion node attachment system, motion node labels, a user's manual, and anatomical diagrams to increase accuracy of motion node placement. In other embodiments, additional features include acceleration data collection and graphical display, rhythmic sound cues for assistance in motion emulation, audio cues for patients with visual impairments, hard copy reports of session results, real time range of motion displays, and a spatial visualization option.

### CROSS-REFERENCE TO RELATED APPLICATIONS

This application claims the benefit of priority to U.S. Provisional Application Ser. No. 61/716,528, filed Oct. 20, 2012, the entire disclosure of which is incorporated by reference herein in its entirety.

### BACKGROUND OF THE INVENTION

Body movement is used daily to perform meaningful occupations and as a result, various occupations require a wide range of body movement. Therefore, understanding normal movement required to complete occupations enhances clinical practice by contributing to the development of biomechanical, adaptive, and rehabilitative devices.

To provide effective intervention, medical professionals need to have a general knowledge of normal body movement. For example, surgeons can use information on normal movement to determine the range of motion (ROM) needed for specific joints to function optimally. Occupational therapy clinicians can also use information on normal movement as a guide to assist in developing applicable patient goals and interventions. Thus, healthcare professionals apply real-time motion capture systems in orthopedic and neurological rehabilitation to engage and retrain patients in motor control rehabilitation. In addition to real-time motion capture technology, current practices of motor control rehabilitation also include the utilization of subjective and objective assessment tools.

Currently, limited data exists on the normality of upper extremity movement. Research measuring normal movement of the upper extremity has not been well established due to the complexity of anatomical structures, large variation of movement during activities, and lack of a standardized method (Murray & Johnson, 2004; Van Andel, Wolterbeek, Doorenbosch, Veeger, & Harlaar, 2008).

Thus, a need exists for a system and method for a reliable and valid tool to measure upper extremity movement to contribute to the knowledge of normal body movement, and ultimately for a low-cost motion capture tool that is more available to clinicians and patients. The present invention satisfies this need.

### SUMMARY OF THE INVENTION

The present invention includes an electronic motion assessment tool. The tool includes a plurality of motion nodes, wherein each motion node comprises an accelerometer, gyroscope, and magnetometer. The tool also includes a means for attaching the motion nodes to at least two joint regions of a subject. In one embodiment, the tool includes an Electronic-Movement Tracker (E-TRK). In another embodiment, the tool includes an Electronic-Movement Logger (E-LOG). In another embodiment, the tool includes a wearable computing device for logging joint movement of the subject. In another embodiment, the tool includes a motion node placement guide. In another embodiment, the plurality of motion nodes record range of motion data. In another embodiment, the tool includes means for graphing rotation, flexion, and abduction motions of a targeted joint in real time. In another embodiment, the tool includes means for generating rhythmic sound cues for assistance in motion emulation. In another embodiment, the tool includes means for measuring frequency, amplitude and smoothness of movement. In another embodiment, the measured movement is used in the assessment of tremor. In another embodiment, the measurement is on a graduated scale. In another embodiment, the tool includes means for generating audio cues for patients with visual impairments. In another embodiment, the tool includes means for generating hard copy reports of session results. In another embodiment, the tool includes means for generating real time range of motion displays. In another embodiment, the tool includes means to emulate a previously recorded motion. In another embodiment, the tool includes means for interactive spatial visualization and emulation exercise. In another embodiment, the visualization and emulation exercise further comprises a ball image. In another embodiment, the ball image translates up/down, left/right and rotates in response to joint rotations flexio/extension, ab/adduction and internal/external rotation, respectively. In another embodiment, the tool includes means to assess movement via a comparison of emulated motion to target with respect to deviation from the target rotations.

The present invention also relates to a method of assessing tremor of a body part of a subject. The method includes the steps of positioning at least one motion node on or adjacent to the body part of the subject to be assessed, wherein the at least one motion node measures frequency, amplitude and smoothness of movement on a graduated scale, and assessing tremor of the body part based on the measured values.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is an image of the attachment equipment for the E-MAT system. Pictured are two motion nodes, and the necessary moleskin, Tubigrip sleeves, and double sided tape for attachment.

FIG. 2 is a series of images showing motion node attachment, with one node on the dorsal aspect of the forearm, avoiding the wrist (bottom right image), and a second node proximal to the elbow, between the lateral and medial epicondyle of the humerus (left image). The completed attachment is shown in the upper right image.

FIG. 3 is a set of images of the E-TRK feature of the E-MAT system measuring, top image: rotation (pronation and supination), and bottom image: flexion (and extension) of the forearm.

FIG. 4, comprising FIGS. 4A and 4B, is a set of screen shots of the system software demonstrating the set up of E-LOG.

FIG. 5, comprising FIGS. 5A, and 5B, is a set of screen shots of the system software demonstrating the set up of E-TRK.

FIGS. 6 and 7 are screen shots of the system software demonstrating setting Zero.

FIG. 8 is a screen shot of the system software demonstrating the re-naming of graphs.

FIG. 9 is a screen shot of the system software demonstrating the selection of the amount of time to collect motion.

FIG. 10, comprising FIGS. 10A through 10C, is a set of screen shots of the system software demonstrating Motion Emulation.

FIG. 11, comprising FIGS. 11A and 11B, is a set of screen shots of the system software demonstrating Saving Data After Motion Collection.

FIG. 12, comprising FIGS. 12A and 12B, is a set of screen shots of the system software demonstrating additional option in the E-TRK menu required for acceleration data collection (A) and an acceleration menu that will appear on screen when user wants to collect acceleration data. Average amplitude and frequency appear on a dial, and maximum and minimum acceleration and frequency are printed in text boxes. Samples should be taken every three seconds (B).

FIG. 13 is an acceleration flow chart.

FIG. 14, comprising FIGS. 14A and 14B, is a set of screen shots of the system software demonstrating an Emulate menu with an option to include Audio cues (FIG. 14A) and an Audio Emulation menu (FIG. 14B) that allows the user to select which motion they would like to accompany change in pitch and/or change in volume.

FIG. 15 is a flow chart of changing pitch and volume audio cues.

FIG. 16, comprising FIGS. 16A and 16B, is a set of screen shots of the system software demonstrating an Emulate menu for Rhythmic Cues (FIG. 16A) and a Metronome menu for Rhythmic Cues (FIG. 16B) that allows a user to enter desired beats per minute, length of cycle, and beats per cycle.

FIG. 17 is a flow chart of rhythmic cues in motion collection.

FIG. 18, comprising FIGS. 18A and 18B, is a set of screen shots of the system software demonstrating a Collect Motion menu including the option for maximum ROM data collection (FIG. 18A) and a GraphForm display including collection of Maximum ROM data, shown in degrees (FIG. 18B).

FIG. 19 is a flow chart of range of motion maximum angles.

FIG. 20, comprising FIGS. 20A and 20B, is a set of motion node labels.

FIG. 21 is a chart illustrating the percentage of time that subjects engaged in areas of occupation.

FIG. 22 is a graph illustrating the percentage of time in ranges of forearm rotation.

### DETAILED DESCRIPTION

It is to be understood that the figures and descriptions of the present invention have been simplified to illustrate elements that are relevant for a clear understanding of the present invention, while eliminating, for the purpose of clarity, many other elements found in motion capture systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The understanding of anatomy and biomechanics of the upper extremity (UE) are necessary to evaluate, diagnose and treat UE pathologies (Bryce & Armstrong, 2008; Zimmerman, 2002). Biomechanics refers to the mechanics of the human body, which helps clinicians analyze body movement (Smith, Weiss, & Lehmkuhl, 1999). Biomechanical knowledge guides clinicians in selection of tasks and intervention strategies during the rehabilitation process (Karagiannopoulos, Sitler, & Michlovitz, 2003; Murray &Johnson, 2004). In addition, data on biomechanics can be used to provide a reference for the determination of disability and impairment (Money, Askew, & Chao, 1981).

An aspect of biomechanics that is necessary to understand and record normal movement is knowledge of ROM. ROM refers specifically to an amount of degrees that a joint moves (Radomski & Trombly, 2008), which provides clinicians with an objective, quantifiable measure of the capability of a joint (Ryu, Cooney, Askey, An, & Chao, 1991). Information on ROM contributes to the understanding of normality, abnormality, and impairment (Money et al., 1981).

Multiple factors contribute to the limited understanding of UE biomechanics within the field of occupational therapy. Clinicians have minimal biomechanical knowledge of the UE due to a limited number of current studies examining the UE biomechanics (Buckley, Yardley, Johnson, & Cams, 1996). Beyond this lack of evidence, the biomechanics of the UE are challenging for researchers to investigate because of the complexity of the anatomical structures of the UE (Van Andel, Wolterbeek, Doorenbosch, Veeger, & Harlaar, 2008). Many factors such as age, sex, and hand dominance (Rickert, Burger, Gunther, & Shulz, 2008), contribute to the disparity and variations of normative data of the UE (Shaaban, Pereira, Williams, & Lees, 2008). Additionally, the variety of movement used during task performance contributes to the complex analysis of the UE (Murray & Johnson, 2004). For example, multiple studies have omitted wrist movement (Murgia, Kyberd, Chappell, & Light, 2004) and joint angles (Magermans, Chadwick, Veeger, & van der Helm, 2005) required to perform daily activities. Furthermore, there is a lack of methods and protocols established to analyze and quantify movement of the UE (Henmi, Yonenobu, Masatomi, & Oda, 2006; Petuskey, Bagley, Abdala, James, & Rab, 2007; Van Andel et al., 2008). Specifically, there is documented opinion that there is a limitation in the methods for measuring forearm rotation (Rickert et al., 2008). As a result, little quantitative data exists related to the UE ROM and biomechanics while completing everyday tasks (Buckley et al., 1996).

Determination of the normal amount of motion needed to complete daily activities aids clinicians in evaluation, diagnosis, and outcome measures (Magermans et al., 2005; Petuskey et al., 2007; van Andel et al., 2008). Investigating and understanding the ROM required to perform daily activities provides clinicians with a more detailed and real picture of the patients' capabilities and impairments (Murgia et al., 2004) however, collecting that data is problematic.

In order to obtain an accurate representation of normal body movement during daily activities, data should be collected in the subjects' natural environment. Research shows that there is a difference in patient performance between the laboratory and the subjects' natural environment (Coley, Jolles, Farron, and Aminian, 2008). Therefore, information on ROM during specific daily activities in a laboratory environment does not reflect actual body movement during daily activities. To address this concern, ambulatory systems, such as accelerometers, allow monitoring of subjects in their usual environment with minimal interference (Coley et al., 2008). Due to accelerometers portability and low weight, they do not require subjects to be in a laboratory environment while collecting data.

While performing research on the UE, it is important to look at the supporting structures including the forearm, hand, elbow, and shoulder. Positions of supporting structures of the UE have a significant impact on UE ROM (Magermans et al., 2005; Shaaban et al., 2008), which enables performance of daily activities (Brumfield & Champoux, 1983). Specifically, there is a reciprocal relationship between the range of forearm rotation and the degree of elbow flexion (Shaaban et al., 2008). The relationship between positions of body structures exemplifies the importance of simultaneously measuring ROM of the elbow and forearm when determining the normal amount of motion needed to complete daily activities.

Elbow, forearm, and wrist pathologies impact the ROM of pronation and supination, making it important for clinicians to assess and restore pronation and supination of the forearm (Rickert et al., 2008). Reliable methods for assessing pronation and supination are important for clinical identification of impairments, functional limitations, and monitoring efficacy of interventions during the rehabilitation process (Karagiannopoulos et al., 2003). Current clinical standards stress the use of functional methods for measuring and restoring physical capacity (Karagiannopoulos et al., 2003).

Data on UE ROM of healthy subjects' performance of daily activities enable clinicians to record and compare pathological UE movements with normal movements (Petuskey et al., 2007). These comparisons can help to identify compensatory strategies and functional improvement after surgical interventions (Magermans et al., 2005). Information on UE ROM during daily activities, can be used to help clinicians evaluate individuals' capabilities (Murgia et al., 2004), activity limitations (Schasfoort, Bussmann, & Stam, 2002), impairments (Money et al., 1981) and outcomes during treatment (Petuskey et al., 2007).

### Electronic-Movement Analysis Tool

The present invention, certain embodiments of which are referred to herein as an electronic movement analysis tool (E-MAT), is a low cost motion capture system for clinical utilizations, such as for upper-extremity (UE) motor control rehabilitation. As contemplated herein, the present invention records range of motion data and graphs rotation, flexion, and abduction motions of a targeted joint in real time. In certain embodiments, the hardware of the system may include a simple motion node attachment system, motion node labels, a user's manual, and anatomical diagrams to increase accuracy of motion node placement. In other embodiments, additional features include acceleration data collection and graphical display, rhythmic sound cues for assistance in motion emulation, audio cues for patients with visual impairments, hard copy reports of session results, real time range of motion displays, and a spatial visualization option.

E-MAT is a system generated from small, lightweight Micro-Electric-Mechanic System (MEMS) inertial sensors, or motion nodes, with an integrated magnetometer, gyroscope, and/or accelerometer used to collect major motions of joints. In certain embodiments, the E-MAT is comprised of two features called the Electronic-Movement Tracker (E-TRK) and the Electronic-Movement Logger (E-LOG). The E-TRK graphs and emulates motions in real time, providing a visual of the motions of a targeted joint, and the E-LOG collects and stores numerical data from the motion of the targeted joint in Microsoft Excel spreadsheet for further analysis in order to track progress over time.

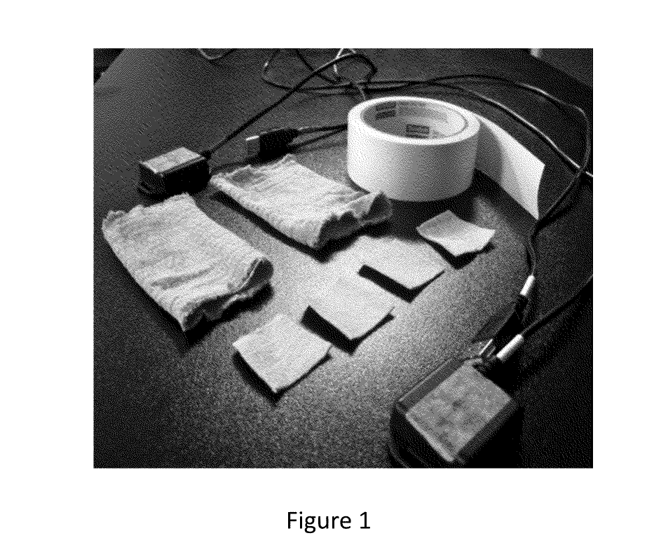

The motion nodes are the integral pieces of the E-MAT system, providing the data for which the computer software records, translates and displays, as described and demonstrated herein. Exemplary motion nodes and attachment equipment are illustrated in FIG. 1. The system may collect flexion and extension, rotation, and abduction movements. The tri-axial accelerometer works within the motion nodes to collect the acceleration of each node with respect to the earth. The gyroscope defines a set of coordinate systems that rotate with the patient's movements. Finally, the magnetometer within the motion node establishes a fixed coordinate system with respect to the earth, preventing drift in data collection. Preventing drift in data collection is important because it allows a motion to start and end at the same zeroed position after back and forth motion. All motions are collected and graphed as a result of these three instruments working in tandem within the motion node.

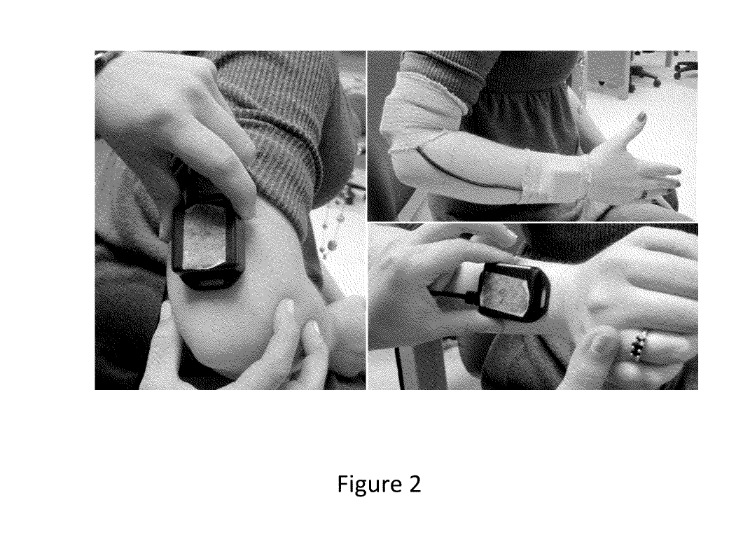

In one exemplary embodiment, the attachment system may include Moleskin and double-sided tape instead or Velcro straps to initially secure the motion nodes to the patient. Two pieces of approximately 1″×1″ Moleskin were stuck together by one 1″×1″ piece of double sided tape. This created a “double-sided Moleskin” and allowed for easy attachment of the motion node to a patient. The motion nodes were then covered with Tubigrip to provide slight compression and security to the motion nodes. Moleskin may be used because it is designed to adhere to skin, and by creating double-sided Moleskin one can apply and remove the motion nodes easily and quickly from a patient as well as replace the double-sided Moleskin after each patient use. The completed attachment of the E-MAT with such an attachment system is shown in FIG. 2.

As contemplated herein, the wires may be designed to permit a wide range of forearm motions. This can be accomplished by: 1) Attaching both the parent and child motion nodes via double-sided Moleskin to the patient; 2) Applying Tubigrip around the parent motion node making sure the wire from the child node runs along the length of the arm. Run the remaining wires of the parent and child nodes around the back of the neck and drape them over the opposite shoulder of the arm attached to the motion nodes; 3) Applying Tubigrip to the child node; and 4) Gently feed the wires through the Tubigrip around the parent node to give the patient more slack and adjust according to the patient's size. A patient with a longer arm span will need more slack. Labels may be used for the motion nodes that define each axis, such as with different colors on a white sticker.

### System Software

The software measures the position of one node with respect to the other. One motion node is denoted as the parent node and the other the child node. The software measures the position of the child node with respect to the parent node. When applied to the forearm, the parent node is the node positioned above the elbow, and the child node is the node positioned on the dorsal surface of the forearm. As contemplated herein, the software has the ability to collect a motion for a set amount of time and graph the motion of the targeted extremity in real time to collect the Range of Motion (ROM) of a targeted extremity. It also has the ability to emulate a motion so that the patient can practice and repeat desired movements for improved accuracy, and so the therapist can initiate relearning of motor control in total movement patterns. A Root Mean Square (RMS) score is revealed after motion emulation which determines the amount of error during motion repetition. During emulation, the computer software has the ability to change the pace of the motion so motions can be repeated at different speeds to assist in rehabilitation. Furthermore, a large deviation can be administered so that the RMS score is better, increasing patient motivation.

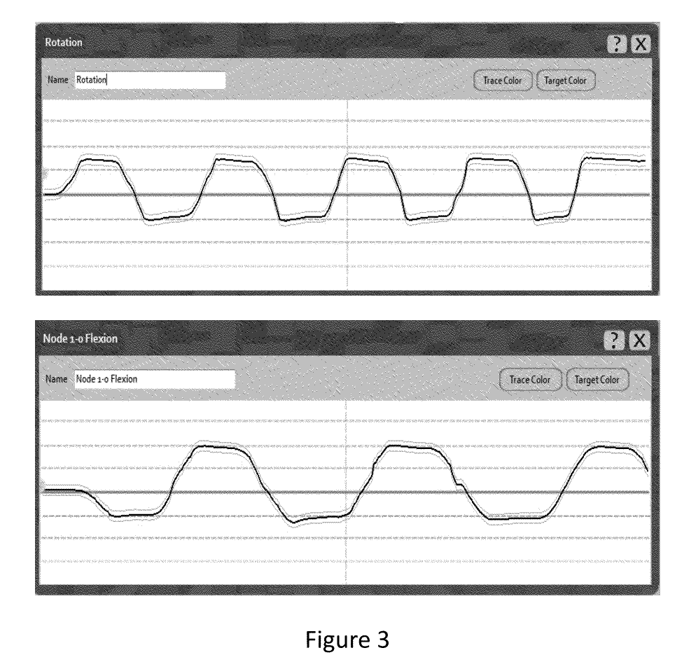

The graphical output of what the patient and clinician sees for a simple movement of rotation and flexion/extension of the forearm is depicted in FIG. 3. The two boundary lines shown on each graph represent the deviation set during motion emulation, acting as an envelope of allowable motion during emulation. The center lines tracking generally within the boundary lines represent the movement of the targeted extremity.

In other embodiments, the software may include additional features. For example, the software may include condensed graphs into one spatial representation of movement. By having the patient focus on one moving object rather than multiple moving objects, they can more accurately perform motions and emulations. The cursor will rotate clockwise or counterclockwise with rotation and supination. There will be a 1:1 ratio between the degrees the patient rotates and the degree the cursor rotates. The cursor will move up and down the same way it currently does with flexion and extension. The cursor will initially be 3″ in diameter and will shrink and enlarge as the patient abducts. 45 degrees will cause a 1″ diameter change of the cursor. In another example, a metronome may be added to assist patients during motion emulation. Adding a metronome that can vary in tempo may help the patient stay in phase during emulation. In another example, the software may utilize accelerometers by displaying acceleration amplitude and frequency. Capturing acceleration data allows the E-MAT to determine acceleration amplitude and frequency, and smoothness of motion. It also allows the E-MAT to be more applicable to patients suffering from Parkinson's disease.

As contemplated herein, logic algorithms specify the goals of a specific feature, outline new design for menu appearances, as well as what the feature should do at each step in the program. In certain embodiments, these additional features make the E-MAT applicable for therapy. The logic algorithms include, by non-limiting example, utilization of accelerometers and ability to visually display acceleration data. This may allow the E-MAT to measure the smoothness of a motion and may extend its application to assist in treatment of a patient with Parkinson's disease. For example, a new graph may appear with two displays. These displays may look similar to a speedometer. One may show the amplitude while the other shows the frequency in Hertz. Furthermore, the maximum and minimum amplitude and frequency may be displayed. Samples may be taken from the motion at various time points, such as at every three seconds. In other embodiments, the logic algorithms display the maximum range of motion for each type of movement in degrees. This feature is aimed the E-MAT's use as an initial assessment tool. This feature is designed to replace the goniometer because of its increased accuracy and more precise measurements. For example, it may create a new graph with added label “Max ROM” and added text field that displays the maximum angle recorded during motion. These may be located between the “Name” text field and the “Trace Color” button. The text field may update automatically throughout data collection if a new maximum angle occurs. In another embodiment, the logic algorithms may include addition of rhythmic cues (i.e. metronome). Rhythmic cues are designed to assist the patient in the E-MAT's emulation feature. This may assist the patient in motion emulation during rhythmic motions. In another embodiment, the logic algorithms may include addition of elevating and diminishing pitch during movement. The feature is designed to assist patients for use of the E-MAT for patients with visual impairments. Other features may include, without limitation: 1) Ability to save and load data into the E-MAT system and Microsoft Excel after motion collection and emulation; 2) Ability to collect and display acceleration amplitude and frequency; 3) Ability to display the maximum range of motion during motion collection; 4) Addition of rhythmic cues for assistance in motion collection and emulation; 5) Addition of varying pitch and volume with movement for patients with visual impairments; and 6) Spatial visualization options.

When preparing the motion nodes and graphs, careful attention to the following steps will ensure that data is collected and graphed properly. Because the motion nodes measure movements with respect to each other, choosing the proper parent and child node is very important for accurate data collection. In the following exemplary embodiment, the program may be ready to run and real time motion capture can begin.

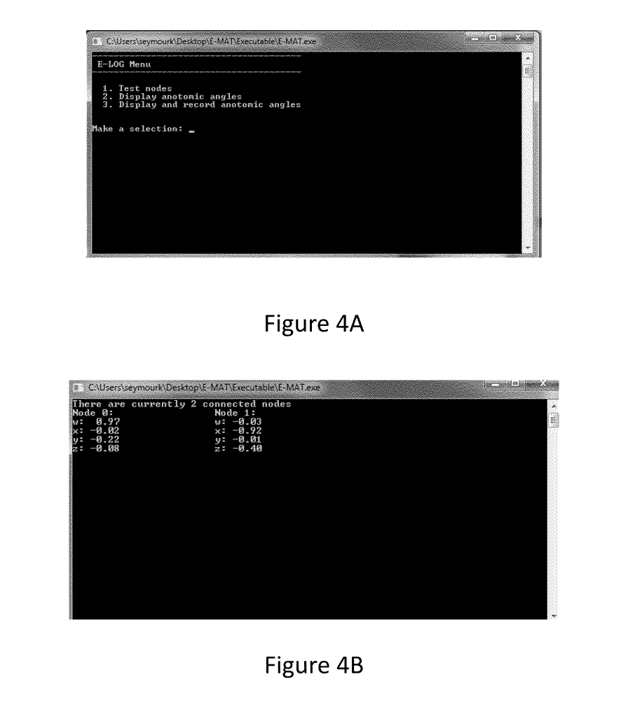

To set up E-LOG: Select the E-MAT icon from the Executable folder. This will open the program with an option of E-LOG or E-TRK. Select E-LOG for system set-up. Three options will appear on-screen, as shown in FIG. 4A: Press the number 1 and then press enter. The screen will appear as shown in FIG. 4B. Moving one node at a time will change the values of one column. After moving all motion nodes one at a time, the heading of each column determines a number that coincides with each motion node.

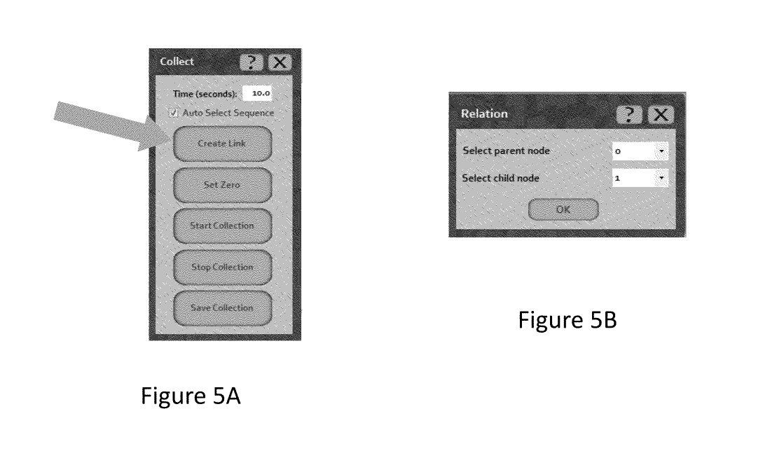

To set up E-TRK: Select the E-MAT icon from the Executable folder. This will open the program with an option of E-LOG or E-TRK. Select E-TRK for graphical program set-up. A pop-up screen will appear offering the choices of Collect Motion, Load Motion, or Emulate Motion. Select Collect Motion, and do not close this window. A toolbar will appear like the one shown in FIG. 5A. Start by selecting Create Link. After selecting Create Link, a new screen will appear like the one shown in FIG. 5B. This is where the parent and child nodes will be defined. Recall the numbers of each motion node from E-LOG Set-Up, Step 3. Assign the most proximal motion node number as the parent node, and the most distal motion node number to the child node. Press OK.

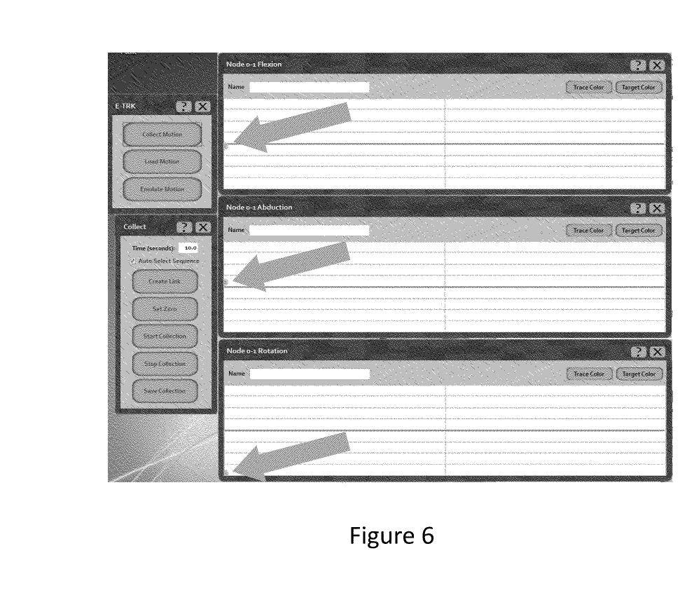

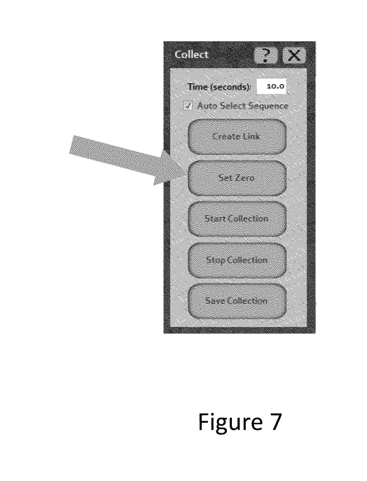

Setting Zero: Three graphs labeled Flexion, Rotation, and Abduction should now be on screen. Move each motion node and notice the blue guide ball moving up and down, as pointed out in FIG. 6. If each guide ball moves, your system should be working properly. Position the client in the Set Zero position described for a particular alignment. When ready to zero, select Set Zero on the toolbar as shown in FIG. 7. The blue guide balls should now all be positioned at zero on each graph.

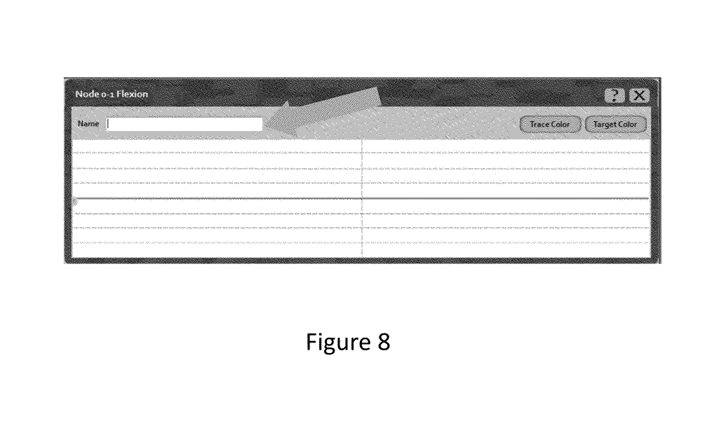

Re-Naming Graphs: After zeroing, the titles of each graph will not necessarily match up to the corresponding motion. First isolate a rotation movement and observe the blue guide balls. Rename the graph with the moving blue guide ball “Rotation” using the place shown in FIG. 8. Repeat process for flexion and abduction motions. Isolate a flexion motion followed by an abduction motion and observe the blue guide balls. Rename the graph with the moving blue guide ball “Flexion” and “Abduction” respectively. If any motions are unnecessary for your specific study, close out of the designated graph. After following all of the steps described above for E-LOG Set-Up, E-TRK Set-Up, Setting Zero, and Re-Naming Graphs, collection motion can be started.

Data Collection allows for real time motion capture of a specific or desired motion based on the set-up and alignment of the motion nodes. After following these steps for collecting motion, one can proceed onto either emulate a specific motion or analyze the data.

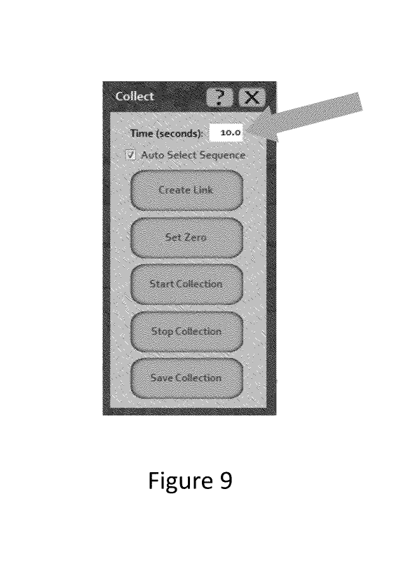

Before beginning to graph motions, select the amount of time you would like to collect motion for. The amount of time per motion can be changed for each new trial by entering a number into the space in the toolbar, shown in FIG. 9. Select Start Collection. The timer will immediately begin and the motion will begin graphing. Should the user ever want to stop a motion in the middle of collection, simply press Stop Collection in the toolbar. If desired, select Save Collection for access at a later time or further analysis. After data is collected and graphed, it can be saved, loaded, emulated, or analyzed for future use.

Motion Emulation allows a client to repeat a motion for repetition and muscle memory, to improve on functional tasks, compare to a desired motion, or many more applications. It can be a very helpful and useful feature after following the steps detailed below.

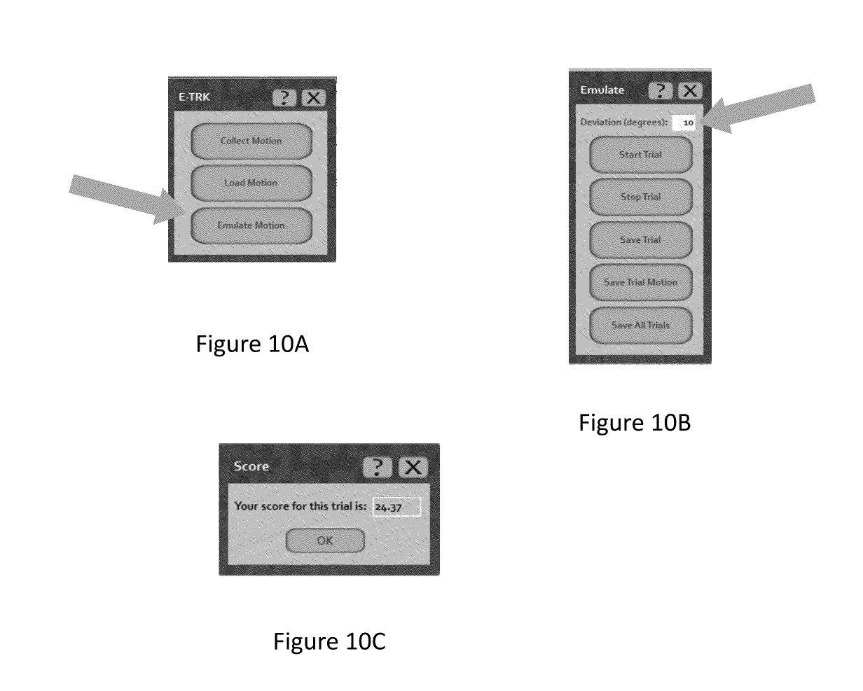

Before motion emulation can begin, make sure that the desired motion for emulation is already graphed on the graphs on screen. This can be done in two ways: 1) Continuing directly onto Emulate Motion after collecting motion, so the motion is still shown on the graphs, and 2) Select Load Motion from the original toolbar, as shown in FIG. 10. This will allow previous motions to be loaded for new motion emulation. After the motion is ready for emulation, continue following these steps: 1) To being motion emulation, select Emulate Motion in the first pop-up screen of FIG. 10A; 2) A new toolbar will appear like the one pictured in FIG. 10B. First select an allowable deviation for motion emulation. This will create an envelope that the client can stay within and still receive a good score at the end of motion emulation. Many times, a high deviation could be beneficial, especially for clients just beginning therapy; 3) The amount of time for the motion can also be altered if desired. Return to Step 1 in Data Collection for how to change the amount of time. This will slow down or speed up the desired motion by stretching it out or shrinking it down; 4) Select Start Collection, where motion emulation will immediately begin. Encourage the client to follow the graphs and the motion as closely as possible; and 5) After the allotted time for motion emulation is complete, a RMS Score will pop up in a new window. The closer to zero the number is, the better the motion emulation was. An example of this screen is shown in FIG. 10C. This step completes motion emulation. Repeat motion as many times as desired, selecting Start Collection to begin each new trial, or save the emulated motion by selecting Save Motion in the Motion Emulation toolbar. If desired, after saving data, continue on to analyze the collected or emulated data.

Data from the collected motions can be saved from E-TRK and then accessed through Microsoft Excel. Analysis can be conducted with numerical data as desired on a specific basis. To save the motion, follow these simple steps. Trials can be saved both after initial motion collection and after motion emulation. The process is similar, but will be mapped out separately in this manual.

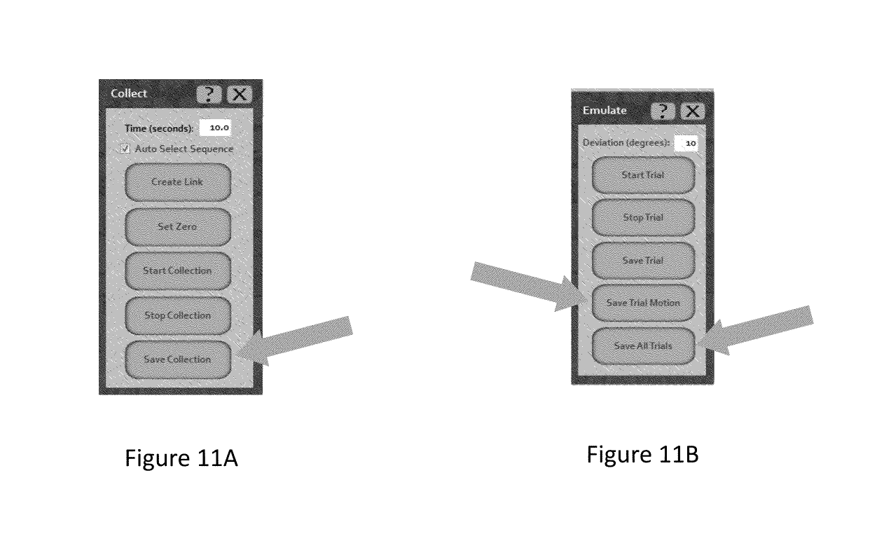

Saving Data After Motion Collection: In the Collect motion toolbar, select Save Collection as shown in FIG. 11A, and Save motion as a .csv file, and then it can be opened in Microsoft Excel.

Saving Data After Motion Emulation: In the Emulate motion toolbar, pictured in FIG. 11B, select: Save Trial Motion if saving only one trial is desired, or Save All Trials if saving repeated trials is desired. Save motion as a .csv file, and then it can be opened for analysis in Microsoft Excel.

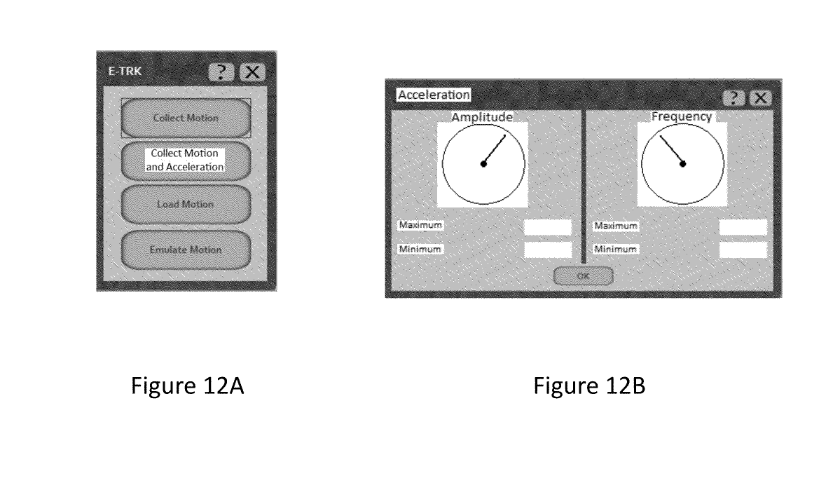

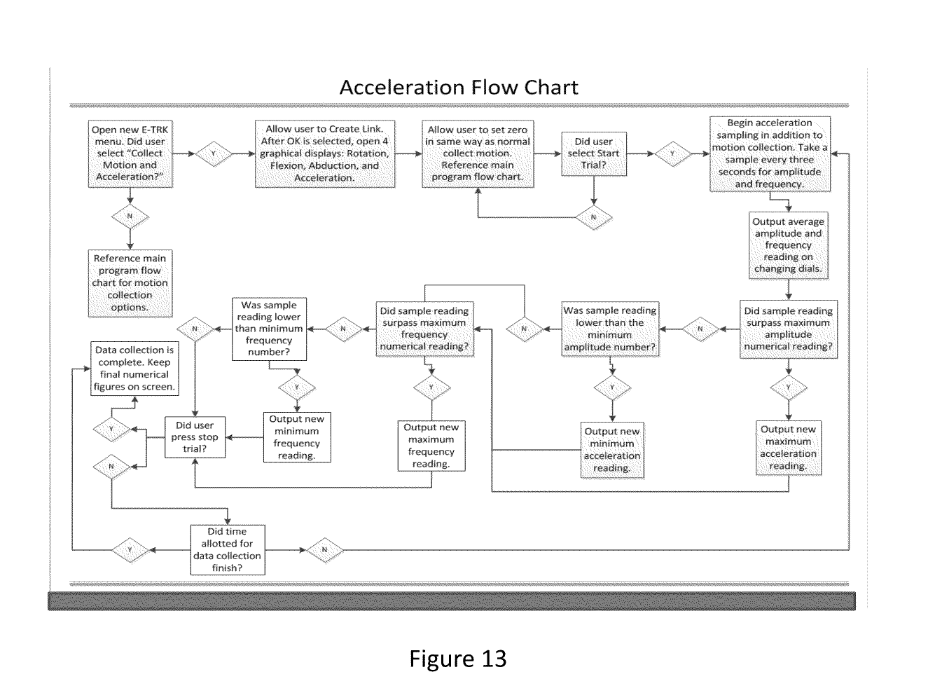

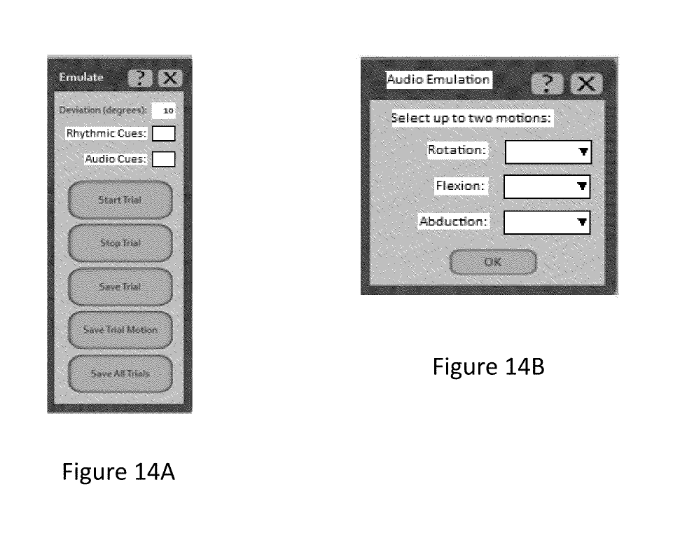

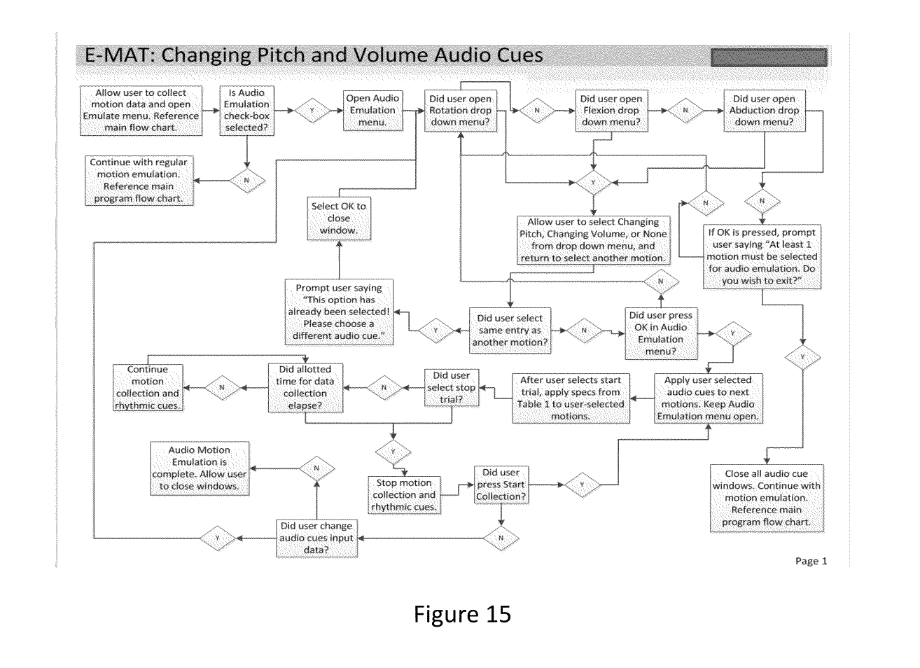

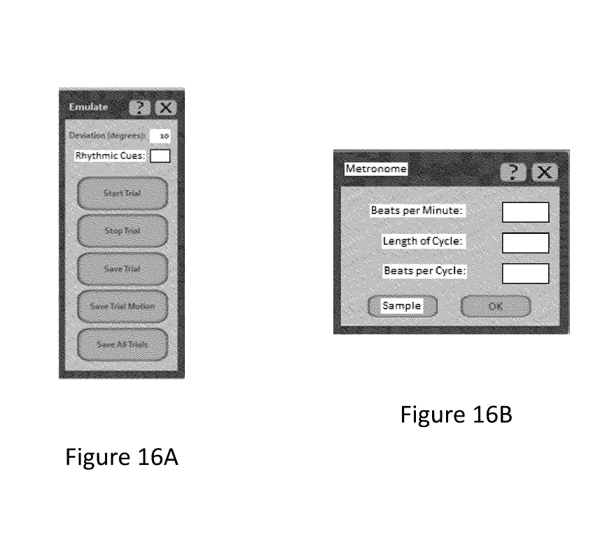

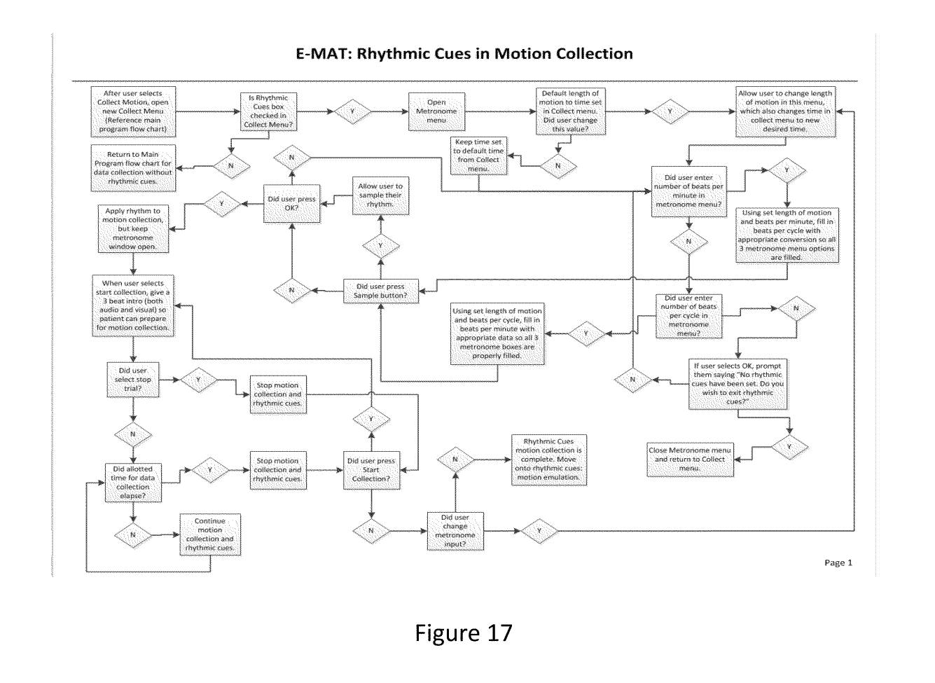

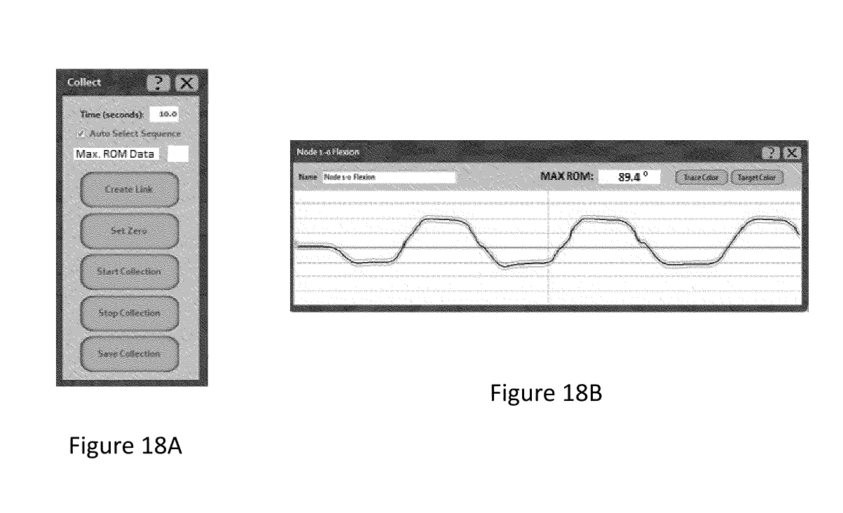

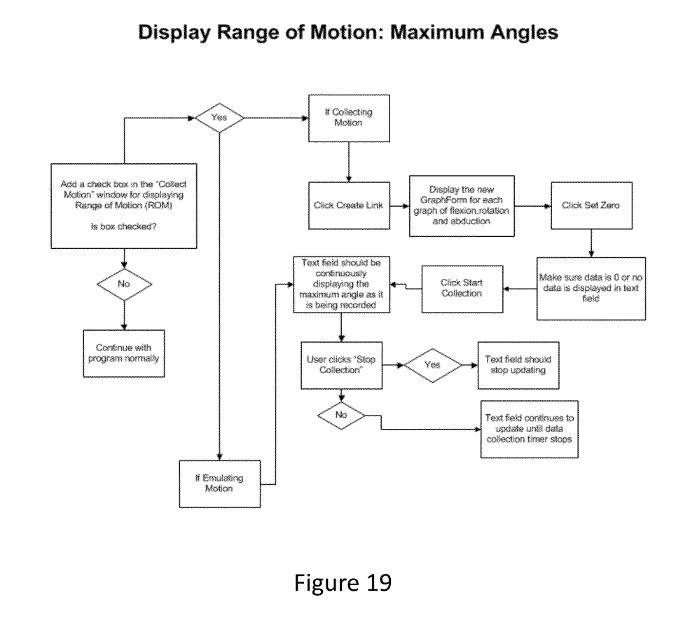

Further, as illustrated in FIGS. 12-19, the system has the ability to capture acceleration data. The purpose of this feature is to collect amplitude and frequency of acceleration in a patient. This feature may be useful for patients with tremors or other involuntary movements. From this collection, the average amplitude and frequency of acceleration will appear on a dial, and the maximum and minimum amplitude and frequency will appear in text boxes. A sample should be taken every three seconds during motion collection for a new average output, and a possible new max/min output. The data should have the ability to be saved to track progress of a patient over time.

| | Pitch during | Frequency of | Decibel Level |
|---|---|---|---|
| Position on Graph | Motion | Note (Hz) | during Motion (dB) |
| Third positive line | C | 130.81 | 20 |
| Second positive line | G | 392 | 30 |
| First positive line | E | 329.63 | 45 |
| Center Line | Middle C | 261.63 | 60 |
| First negative line | G | 220 | 75 |
| Second negative line | E | 174.61 | 90 |
| Third negative line | C | 523.25 | 100 |

Varying pitch and volume audio cues: The purpose of changing pitch audio cues is to assist a patient in motion emulation, specifically for patients with visual impairments. Changing pitch and volume should indicate a change in motion. The system allows the client to choose to isolate a motion or perform different motions together. The program should also allow the client to choose which motion is given a changing pitch and/or a changing volume. The above table indicates the desired pitch and volume level during motion emulation. The user should be able to hear a defined difference for both pitch and volume for this tool to be effective.

Rhythmic cues in motion collection: The purpose of E-MATs rhythmic cues feature is to assist patients in ROM activities during motion collection and emulation. Rhythmic cues from a metronome in the program will allow patients to more accurately perform motions.

Before the motion collection begins, visual and audio cues may be given as a warning that the motion is about to begin. This will allow the user to see and hear the rhythm prior to motion collection. A “3,” “2,” “1” should appear on a pop-up screen at the same tempo as the set rhythm. Three beats at the same rhythm should accompany the visual cues. After that, the pop-up window should disappear and the rhythm should continue right into motion collection.

Display maximum ROM: Create a new GraphForm with added label “Max ROM” and added text field that displays the maximum angle that was recorded during motion. These should be located between the “Name” text field and the “Trace Color” button. The text field should update throughout data collection if a new maximum angle occurs. This feature will allow the patient to numerically see their ROM ability and allow for easier print outs of the graph and data.

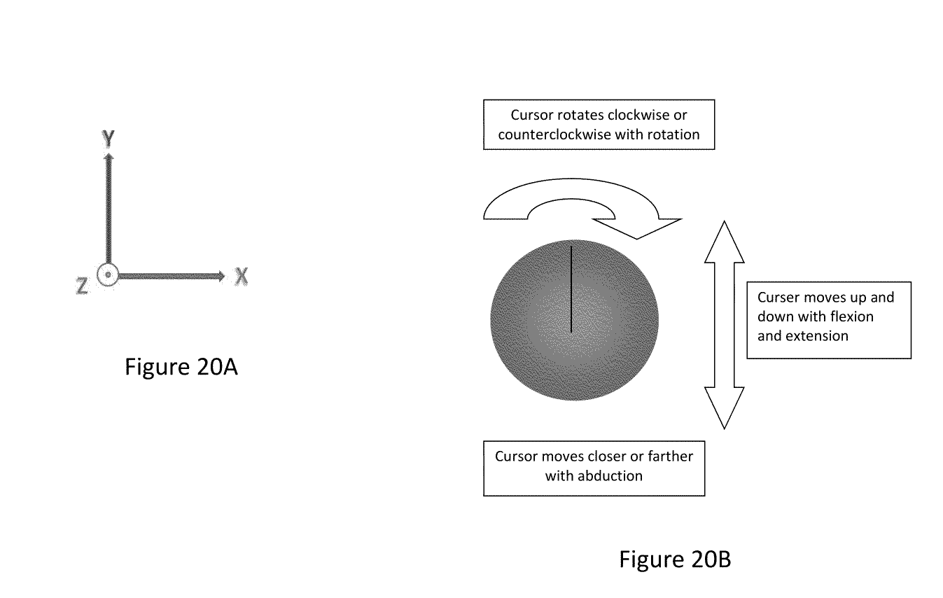

Motion node labels were created with a proper fit for the motion node. They are sticker labels and simple to apply to the motion node. The motion about the X axis is rotation, the motion about the Y axis is abduction, and the motion about the Z axis is flexion. A sample of what the motion node stickers is shown in FIG. 20.

Generally, the system of the present invention may operate on a computer platform, such as a local or remote executable software platform, or as a hosted internet or network program or portal. In certain embodiments, only portions of the system may be computer operated, or in other embodiments, the entire system may be computer operated. As contemplated herein, any “computer platform” may be operable from any computing device as would be understood by those skilled in the art, including desktop or mobile devices, laptops, desktops, tablets, smartphones or other wireless digital/cellular phones, televisions or other thin client devices.

For example, the computer operable component(s) of the system may reside entirely on a single computing device, or may reside on a central server and run on any number of end-user devices via communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data format between two or more computing devices. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

Further, the communications network may also use standard architecture and protocols as understood by those skilled in the art, such as, for example, a packet switched network for transporting information and packets in accordance with a standard transmission control protocol/Internet protocol (“TCP/IP”). Any of the computing devices may be communicatively connected into the communications network through, for example, a traditional telephone service connection using a conventional modem, an integrated services digital network (“ISDN”), a cable connection including a data over cable system interface specification (“DOCSIS”) cable modem, a digital subscriber line (“DSL”), a T1 line, or any other mechanism as understood by those skilled in the art. Additionally, the system may utilize any conventional operating platform or combination of platforms (Windows, Mac OS, Unix, Linux, Android, etc.) and may utilize any conventional networking and communications software as would be understood by those skilled in the art.

Further, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. For example, a system administrator may allow for administration at one or more levels, such as at an individual user (patient) level, a healthcare professional level, or at a system level. A system administrator may also implement access or use restrictions for users at any level. Such restrictions may include, for example, the assignment of user names and passwords that allow the use of the present invention, or the selection of one or more data types that the subservient user is allowed to view or manipulate.

As mentioned previously, the system may operate as application software, which may be managed by a local or remote computing device. The software may include a software framework or architecture that optimizes ease of use of at least one existing software platform, and that may also extend the capabilities of at least one existing software platform. The application architecture may approximate the actual way users organize and manage electronic files, and thus may organize use activities in a natural, coherent manner while delivering use activities through a simple, consistent, and intuitive interface within each application and across applications. The architecture may also be reusable, providing plug-in capability to any number of applications, without extensive re-programming, which may enable parties outside of the system to create components that plug into the architecture. Thus, software or portals in the architecture may be extensible and new software or portals may be created for the architecture by any party.

The system software may provide, for example, applications accessible to one or more users to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system.

The system software may also be a portal that provides, via the GUI, remote access to and from the system of the present invention. The software may include, for example, a network browser, as well as other standard applications. The software may also include the ability, either automatically based upon a user request in another application, or by a user request, to search, or otherwise retrieve particular data from one or more remote points, such as on the internet. The software may vary by user type, or may be available to only a certain user type, depending on the needs of the system. Users may have some portions, or all of the application software resident on a local computing device, or may simply have linking mechanisms, as understood by those skilled in the art, to link a computing device to the software running on a central server via the communications network, for example. As such, any device having, or having access to, the software may be capable of uploading, or downloading, any information item or data collection item, or informational files to be associated with such files.

Presentation of data through the software may be in any sort and number of selectable formats. For example, a multi-layer format may be used, wherein additional information is available by viewing successively lower layers of presented information. Such layers may be made available by the use of drop down menus, tabbed pseudo manila folder files, or other layering techniques understood by those skilled in the art. Formats may also include AutoFill functionality, wherein data may be filled responsively to the entry of partial data in a particular field by the user. All formats may be in standard readable formats, such as XML. The software may further incorporate standard features typically found in applications, such as, for example, a front or “main” page to present a user with various selectable options for use or organization of information item collection fields.

The system software may also include standard reporting mechanisms, such as generating a printable results report, or an electronic results report that can be transmitted to any communicatively connected computing device, such as a generated email message or file attachment. Likewise, particular results can trigger an alert signal, such as the generation of an alert email, text or phone call, to alert an expert, clinician or other healthcare professional of the particular results.

### Therapeutic Applications

As contemplated herein, the E-MAT system may be applied in therapy for patients with neurological conditions, such as patients with stroke and Parkinson's disease. In certain embodiments, the present invention may also be used for orthopedic applications. The E-MAT may also be applied to patients working on active function to activate a flaccid tone, or patients working on improving their gross motor movements.

The present invention may also be applied as an assessment and/or an intervention tool. As an assessment tool, the E-MAT may measure severity and ROM of a patient in the beginning of therapy. The E-MAT may also measure a baseline of where the patient is at the start of therapy and then track change over time through regular data collections. Finally, the E-MAT may measure smoothness of motion as an assessment tool.

As an intervention tool, the E-MAT may be applied to the good side of a patient first in order for the patient to get a feel for the system, then switched to the bad side for comparison. Also, the E-MAT may increase muscle memory through back and forth motion and various ROM activities. Furthermore, the E-MAT may also compare functional tasks to ROM activities on an isolated joint. In addition, the E-MAT may help a patient practice Activities of Daily Living (ADLs) by creating and storing motion, then emulating how it should be done. Finally, implementing a larger deviation during motion emulation may increase patient motivation and confidence.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

The initial prototype study of the E-MAT examined the accuracy and consistency of the MotionNode accelerometers and the designed computer software program to gather range of motion of the forearm. The first generation proved the reliability and validity of the accelerometers and computer software program. In the first generational study, the E-MAT measured upper extremity movement and contributed to the knowledge of normal body movement. Implications were formed in conclusion of the initial prototype study of the E-MAT for a second generational study to further develop the tool for clinical utilization.

The purpose of this study was to develop the initial prototype of the E-MAT into a low-cost, motion capture tool that would be useful in motor control rehabilitation as utilized in the clinic to objectively assess a patient. Since upper extremity motor control rehabilitation is a major aim of recovery in many orthopedic and neurological populations, such conditions were targets in clinical application. The aim of the study was to answer the research question, how can the developing E-MAT system be utilized in motor control rehabilitation of the upper extremity for patients with orthopedic and neurological impairments? The researchers engaged the E-MAT in the saturation stage of tool development through tool demonstration and discussion with experts in the field of motor control rehabilitation to identify ways to adapt the E-MAT measurement tool for clinical utilization.

### Methodology

### Participants

The study engaged three medical professionals with clinical expertise in motor control rehabilitation as the participants of the study. The professionals were selected based on expertise in the field of motor control rehabilitation, location, and availability; therefore, participants were collected through convenience sampling (Portney & Watkins, 2009).

A total of eleven professionals were initially identified and contacted via email to explain the purpose of the research study and request participation to review the tool for further development towards clinical application. Following this request, three professionals agreed in writing to participate. All three participants were medical or rehabilitative professionals from a university hospital. One participant was a neurologist with a specialty in disorders of the nervous system and had practiced in the field for over 35 years. This participant's patient population currently consisted of individuals seeking stroke and brain injury rehabilitation. The second participant was a practicing hand therapist with a background in occupational therapy. This participant's patient population currently consisted of individuals with an array of diagnoses including stroke, congenital abnormalities, head injuries, and cerebral palsy. The third participant was a movement disorder specialist and the director of brain analysis research for neurodegenerative disorders. This participant currently focuses research on rehabilitation for patients with Parkinson's disease. The variety of candidates offered a range of perspectives and ideas on the E-MAT's application to motor control rehabilitation. Participants did not receive compensation but engaged in professional, stimulating discussions of their opinions and experiences with motor control rehabilitation. Participants provided written approval to use his or her name and facility in this study so recognition could be properly awarded.

### Instruments

MotionNodes. MotionNodes are small, lightweight, and low-cost instruments that collect major motions of joints. Each MotionNode in this study was 56 millimeters X 40 millimeters X 15 millimeters in size, weighing 14 grams, costing $1,000 per MotionNode. The E-MAT's MotionNodes have an integrated accelerometer, gyroscope, and magnetometer. Accelerometers provide acceleration and an angular rate of body movement in real-time, gyroscopes measure rotation, and magnetometers measure the orientation with respect to the earth's magnetic field (Bakke & Tokheim, 2010). MotionNodes are instruments that monitor movement with minimal interference and are manageable in normal environments (DeGoede et al., 2011). The MotionNodes measure acceleration along three anatomical axes, (X, Y, and Z rotations) with respect to the earth (DeGoede et al., 2011). Two MotionNodes can be set up in a parent-child relationship for data to be recorded on two joint positions and orientation to each other. Research shows that placement is very critical to maintain reliability of the tool environments (DeGoede et al., 2011). The prototype of the E-MAT identified proper MotionNode placement for elbow measurements as follows: One is placed above the elbow, between the lateral and medial epicondyle of the humerus and the second is attached on the dorsal surface of the forearm, over the distal aspect of the radius and ulna. In addition to MotionNode placement, the arm must be positioned in ninety degrees of elbow flexion, with the forearm in neutral position so that the hand is rotated with the thumb pointed up (DeGoede et al., 2011).

In this study, the MotionNode attachment method was altered from a previous Velcro strap used in the first generation study to a more comfortable set-up including Moleskin between the forearm placement and the MotionNode. Tubigrip were worn on top of each MotionNode to ensure secure attachment and to help with wire management during movement. During our demonstration of the E-MAT, two MotionNodes were attached to the forearm to measure joint movement of the wrist relative to the elbow, specifically flexion and extension, and pronation and supination.

Computer software. The computer software of the E-MAT provides data to determine range and frequency of total movement patterns by measuring the relative position of one MotionNode to the other at a rate of 100-Hz. MotionNodes connect to any computer via USB port. The computer software can collect data from the MotionNodes and graph movements in real-time. For this particular study highlighting the elbow joint and forearm rotation, two graphs were displayed demonstrating flexion and extension of the elbow and pronation and supination of the forearm in motion. In conjunction with the MotionNode's gyroscope, the computer software applies a real-time motion capture system to create a movement trace.

The E-MAT's computer software is composed of two systems, the E-TRK and the E-LOG. The E-TRK graphs movement of the targeted joint in real-time and collects and emulates performed joint movement patterns and immediately displays this information on a graph. This feature visually depicts movement of a targeted extremity reflected on a graph (FIG. 3). The E-LOG feature enables users to save the graph's data points into an Excel document, which is beneficial for measuring progress of the targeted extremity over time.

### Demonstration and Application to Practice

With this equipment, a therapist may initiate relearning motor control in total movement patterns. A possible application of the E-MAT is to create a movement trace wearing the accelerometers. The movement trace appears on the computer screen and the system plots the joint angle in comparison to time for up to two joints in a kinematic chain. The plot can then be saved. Afterwards, the demonstrated movement trace can be emulated with real-time feedback. The plot created by the computer software allows for immediate visual feedback of total movement patterns. During this study, the researchers received professional opinion on further utilization of the software within clinical application through demonstration of the computer software.

### Procedures

Three student researchers, one occupational therapy student and two mechanical engineering students from a comprehensive college facilitated this second-generation prototype of the E-MAT. Following a methodological research design, the researchers aimed “to develop or refine procedures or instruments for measuring variables” (Portney & Watkins, 2009, p. 291). After Institutional Review Board approval from the researchers' institution was obtained with a waiver of informed consent, professionals were contacted via email to probe participation and arrange a convenient meeting time and location. The purpose of meeting was to review the instrument and secure professional opinions about the instrument.

Prior to meeting, the researchers established information needed to properly develop the E-MAT tool. A semi-structured interview was designed with questions to gather all the necessary information from the participants. Both the occupational therapy student researcher and the engineering student researchers independently created a set of interview questions to guarantee coverage of all areas of need. The occupational therapy student focused on therapeutic application while the engineers focused on features of the equipment and software. The researchers combined question sets to create one comprehensive list. Several drafts were created. To further direct the focus of each question, a mock-interview was performed on the researcher advisors to direct the focus of each question. A finalized list of eighteen questions was organized into a logical series to probe participants' professional feedback. These interview questions were sent via email to each participant to review prior to meeting with the researchers. The semi-structured interview questions asked participants to identify rehabilitation techniques currently used in motor relearning, including measurement tools, technological advances, or ways to track data collection. The questions guided discussion about likes and dislikes of the current measurement tool and also inquired perceived application of the E-MAT to clinical utility. The semi-structured interview questions were short, open-ended and written in simple sentences.

Each participant was interviewed in his or her work office at the university hospital. Upon meeting, the researchers initiated semi-structured interviews with the participants about their current experience in motor control rehabilitation to understand their expertise in this field. The semi-structured interview was interjected with tool demonstration of the system's accelerometers and computer software for the participant to have hands-on experience with the E-MAT. After demonstrating the E-MAT, the participant continued to engage in the semi-structured interview to draw their critique of the E-MAT and the tool's utilization and application to motor control rehabilitation. Researchers ensured rapport with participants and encouraged an open environment focusing on professional concerns. Following a methodological approach, the data collected was then analyzed and implications were identified to apply findings for further development.

### Data Analysis

Throughout the semi-structured interview process, each researcher recorded separate notes from the participant's responses to the probed questions. Immediately following each interview, the researchers discussed and documented field notes of major points identified by the participant. Then researchers discussed the participant's response to each question and recorded an organized response based on each of researcher's notes from the interview. After all three interviews were completed, the researchers' collaboratively identified themes for tool development recommended for clinical application in motor control rehabilitation.

### Results

### Application in Therapy

Interviews with each participant relayed valuable information on potential utilization of the E-MAT in motor control rehabilitation of patients with neurological conditions for both assessment and intervention purposes. Participant responses were homogeneous and indicated E-MAT application in therapy for patients with neurological conditions, such as stroke, Parkinson's disease, or multiple sclerosis. Of the participants, the physician focused on E-MAT application for patients with stroke, the movement disorder specialist emphasized integration for patients with Parkinson's disease, and the therapist did not focus on one condition. Responses indicated the E-MAT could specifically benefit patients working on 1) wrist extension or ankle dorsiflexion, 2) active function to activate a flaccid tone, or 3) gross motor movements. All participants indicated the E-MAT was applicable not only for assessment purposes as initially hypothesized by the researchers, but also as an intervention approach. As an assessment, the therapist supported the E-MAT to measure tremors, smoothness of motion, initial severity, or to measure a baseline of range of motion and change over time. The E-MAT could be applied as an intervention approach for the recommended populations to compare an affected extremity versus an unaffected extremity, to compare functional tasks versus range of motion activities of an isolated joint, to practice activities of daily living (ADL) performance, or to increase muscle memory through repetitive motions. For both assessment and intervention application, a suggestion was made to provide visual print out of the graphs from the emulated motions in order to record patient performance.

### Consumer Market

Interviews with each participant led the researchers to several conclusions regarding the E-MAT in the consumer market. First, participants individually expressed in accord that the E-MAT system is user-friendly and simple enough for practitioners to implement and patients to follow. Second, results conclude that the E-MAT's adapted attachment using Moleskin and Tubigrip is simple and effective, to securely place a MotionNode on the patient's forearm. Thirdly, the participants all confirmed that the E-MAT is low-cost; at $1,000 per MotionNode, this system is offered at half of the price of the nearest competitor (APDM: Movement Monitoring Systems, 2012). Also, one participant confirmed the E-MAT system would fit under a minor budgeting expense; she stated, receiving approval for proposals of less than $5,000 is feasible because such investments are considered a minor budget expense at the participant's university hospital.

### Implications and Potential

As contemplated herein, added audio or visual cues could provide positive feedback for the patient. An example was to create a virtual reality program to integrate the patient's movements and increase motivation. Further, audio cues could be added to assist patients with visual impairments by elevating and diminishing pitch based on range of motion. For example, as the patient should raise the forearm, the pitch would become higher and as the patient should lower the arm, the pitch would become deeper. As contemplated is to utilize the acceleration feature of the MotionNode to measure reactive timing, speed, and involuntary movement. Another concept is to use the developing E-MAT system to prove quantifiable effectiveness of interventions, such as adaptive equipment or medications. An example was to compare the performance of a patient with Parkinson's disease's with and without weighted utensils to measure size and influence of tremors on movement; the E-MAT could provide some objective evidence to the effectiveness of such interventions. The E-MAT should be tested as an assessment and intervention tool in future studies. As contemplated herein is a randomized control trial study, using the E-MAT system on acute stroke patients to evaluate the effectiveness for this population. Another implication was to apply the E-MAT to another joint on the body, such as the shoulder, the ankle, the knee, or the hip. Also contemplated herein is to create anatomical diagrams of MotionNode placement on specific joints to increase consistency and reliability.

### Discussion

### Application to Occupational Therapy

As per our participants, this study demonstrated that the developing E-MAT system has utilization in therapy as an objective assessment and intervention tool for patients with neurological conditions. In general, the brain's plasticity is “associated with improvement or rehabilitation of motor skills” (Winkler, Mergner, Szecsi, Bender, & Straube, 2012). Commonly performed in therapy, “neurofacilitation approaches treat patients with neurological impairment” and are “focused on retraining motor control through techniques designed to facilitate and/or inhibit various movement patterns” (Shumway-Cook & Woollacott, 2001, p. 23). Since motor control is affected by the central nervous system, neurological involvement often compromises a patient's motor control (Secoli, Milot, Rosati & Reinkensmeyer, 2011). Within a neurofacilitation approach, “emphasis is placed on the understanding that incoming sensory information stimulates and thus drives a normal movement pattern” (Shumway-Cook & Woollacott, p. 23). As movement patterns are commonly performed in therapy of neurological conditions, the E-MAT can collect data of total movement patterns, the E-MAT may be effective for gathering valuable data. This research demonstrated that the E-MAT may be applied to patients performing range of motion activities and will record changes in motor performance and progress over time.

Not only does the E-MAT have potential for utilization during neurofacilitation approaches, this study also demonstrated that the developing E-MAT system has utilization as a task-oriented approach. “For retraining [motor] control, it is essential to work on identifiable functional tasks rather than on movement patterns” (Shumway-Cook & Woollacott, 2001, p. 25). “A task-oriented approach to intervention assumes that patients learn by actively attempting to solve the problems inherent in a functional task rather than repetitively practicing normal patterns of movement” (Shumway-Cook & Woollacott, 2001 p. 25). Repeating movement patterns provides a patient with memory on the outcome of the movement, called knowledge of results (KR) (Shumway-Cook & Woollacott, 2001). Shown in this research, medical professionals expect the E-MAT to be applicable during task-oriented approaches of interventions, such as practicing activities of daily living (ADLs), comparing functional tasks to range of motion activities, or increasing muscle memory through repetitive task performance.

Within task-oriented approaches, normal movement emerges as an interaction of many systems (Shumway-Cook & Woollacott, 2001). Considering body movement as a system, research found that after a stroke, “intrinsic feedback mechanisms are frequently compromised, so the provision of extrinsic feedback may be beneficial” (Robertson et al., 2009). Extrinsic feedback can be provided through KR as the movement's goal is remembered and repeated. However, knowledge of performance (KP) is an applicable extrinsic feedback approach to the E-MAT's application. For instance, with the E-MAT, as the movements are performed KP feedback is provided to the patient about the movement pattern utilized to achieve the goal (Shumway-Cook & Woollacott, 2001). Other extrinsic feedback provided in the E-MAT tool includes visual aids that display performed movements on a graph. Additional audio and visual cues may improve effectiveness of the E-MAT to provide additional extrinsic feedback for patients. Real-time motion capture technology like the E-MAT provides interactive experiences within physical, natural environments to increase generalizability of performance in activities beyond rehabilitation (Lehrer, Attygalle et al., 2011). However, simplicity should be integrated because a way to maintain motivation is to make sure a patient understands his or her therapy, which can be an issue when using real-time motion captured technology. Research discovered that the easier the visual representation of performance or results, the better a patient could understand therapy (Colombo et al., 2007).

Also, participants in this study articulated the importance of viewing the body as a system and provided expert opinion that the E-MAT may have potential to address reflexes as well as voluntary and involuntary movements. Viewing the body as a system, the World Health Organization demonstrates that neuromuscular and movement-related body functions are comprised of reflexes, voluntary and involuntary movements, and sensation (World Health Organization, 2012). Voluntary movements are classified as “functions associated with control over or coordination of” simple or complex voluntary movements (World Health Organization, 2012). Discussion with the participants led to the platform that the E-MAT could address voluntary as well as involuntary movements. To further understand effectiveness of the E-MAT with involuntary movements, research supports application with the population of patients with Parkinson's disease. For example, a set of accelerometers was used in a study to capture subtle tremor features (Rigas et al., 2012). This study proved effectiveness of accelerometers, similar to those used with the E-MAT system to “successfully: (i) quantify tremor severity with 87% accuracy, (ii) discriminates resting from postural tremor and (iii) discriminates tremor from other Parkinsonian motor symptoms during daily activities” (Rigas et al., 2012). Since tremor detection can be captured with acceleration data (Rigas et al., 2012; Huang et al., 2012; Lewek, Roole, Johnson, Halawa, & Huang, 2009), the E-MAT should begin to utilize the available acceleration feature to record reactive timing, speed, and involuntary movements during performance.

### Example 2

Methodological research is commonly used in instrument development to determine the reliability and validity of an instrument (Portney & Watkins, 2009). A methodological research design was used in this study to examine the reliability and validity of a pilot instrument, using MotionNode accelerometers, to measure functional forearm rotation.

### Participants

Male and female volunteer participants were recruited for this study and were obtained through a convenience sample of Occupational Therapy and Engineering students enrolled at Elizabethtown College. An email explaining the research and asking for volunteers was sent to all Occupational Therapy and Engineering students. All participants signed an informed consent document that explained the research procedure and allowed researchers to disseminate the data.

To protect subjects' confidentiality each subject was assigned to a random letter which was used to label their demographic form, semi-structured interview, and Occupational Questionnaire. This allowed their name to be separated from their personal data. Only the researchers knew which name corresponded to each letter. This information was recorded and kept in a locked room, to which only the researchers had a key. Subjects' demographic information was used solely for the purpose of statistical analysis.

All participants completed a demographic form, which determined their eligibility to participate. The demographic form was developed by the researchers to gather subject information that contributed to the analysis of the data. Inclusion criteria included that subjects had to be 18 years of age, healthy with no known current or previous UE limitation, pain, or injury of the dominant arm. Exclusion criteria included that subjects had current or previous injury, surgery, pain, or limitation of the shoulder, arm, elbow, forearm, wrist, and hand, as well as an allergy to latex. Active range of motion that was not within normal limits of 80°-90° supination and 70°-80° pronation (Smith, Weiss, & Lehmkuhl, 1996) excluded subjects participation. Participants were informed that they could not get the instrument wet or be in contact with factory machinery while wearing the instrument.

### Equipment Used

Accelerometers can be used for the assessment of dynamic activity (Westerterp, 1999) due to their ability to objectively measure body movements and record data to assess physical activities (Chen, Acra, Majchrzak, Donague, Baker, Clemens, & Sun, 2003). The accelerometers used in this study are triaxial MotionNode accelerometers that are manufactured by GLI Interactive LLC in Seattle, Wash. Triaxial accelerometers are electronic devices that measure acceleration along three anatomical axes (X, Y, and Z rotations) (Hale, Williams, Ashton, Connole, McDowell, & Taylor, 2007). The devices used in this study couple the accelerometers with 3 gyroscopes, which measure rotations, and 3 magnetometers, which measure the orientation with respect to the earth's magnetic field. MotionNode accelerometers are designed to be used in biomechanical research as they provide orientation, acceleration, and angular rate of body movement in real-time (Bakke & Tokheim, 2010). The MotionNode accelerometers were chosen based on their price and functionality.

When using more than one accelerometer it is important to consider the comfort of the subjects and the potential interference with physical activity (Westerterp, 1999). Subjects' comfort while wearing an accelerometer is ultimately determined by the site of attachment, size, and weight of the accelerometer (Westerterp, 1999). MotionNode accelerometers are small (56 mm×40 mm×15 mm) and lightweight (14 grams) which made them suitable to our research, since they were attached to human subjects. Consistent placement of accelerometers and incorporation of anatomical reference points such as bony protuberances is essential to ensure data is reliable and valid (Alves, et al., 2009; Hale, et al., 2007). When targeting a specific body movement with an accelerometer, placement is critical, as a 1-2 cm shift in accelerometer location can cause significant data miscalculation (Alves, Sejdić, Sahota, & Chau, 2009; Hale, et al., 2007).

The computer used to run the software was selected based on three primary criteria. It was necessary for the computer to have two USB ports to connect the two accelerometers to the computer. The computer also needed to be small, lightweight, and durable because it was worn by the subjects in a backpack. A long battery life enabled the subjects to wear the equipment for a long period of time without needing to be recharged. The computer needed to be adapted to run while in a closed position. A solid state netbook, ASUS Eee pc 901, with a size of 8.9×6.7×0.8 inches and weight of 2.2 pounds, was chosen based on its ability to meet these needs.

The software provided by MotionNode was not able to meet our specific needs for this biomechanical research. A custom data collection program was created for the accelerometers which allowed us to examine the angle of pronation and supination of the forearm. Programming language C# along with MotionNode's Lua scripts, was used to develop and implement this program on the netbook computer. The program was written to record the Euler angles of the accelerometers. The software collects data from the accelerometers at a rate of 10 Hz (times per second). The Euler angles gathered from this software were converted into degrees of pronation and supination and presented in a histogram through Microsoft® Excel®.

The accelerometers needed to be held in place at the appropriate location on the elbow and forearm. An adjustable, elastic, soft strap that can fit any size arm was developed. The accelerometers were attached to the straps with Velcro®. This allowed the accelerometer to be attached to the proper position on the subjects. Tubigrip™ was placed over the accelerometer and strap to ensure the accelerometers did not move and to keep the wires from getting caught. After trying different combinations and wearing the attachment ourselves we determined that the combination of Velcro® and Tubigrip™ was the most comfortable and least restrictive of arm movement.

### Assessments Used

Goniometers are used for measuring joint motion (Radomski & Trombly, 2008). A goniometer uses a protractor, an axis, and two arms to measure the ROM at a specific joint (Radomski & Trombly, 2008). In order to ensure reliability of the goniometric measurements, the researchers needed to place the axis and arms appropriately and consistently (Radomski & Trombly, 2008). In this study the researchers used appropriate and consistent placement of goniometers by following the guidelines provided by the reference book, Occupational Therapy for Physical Dysfunction. Multiple studies have found the intraclass correlation coefficient of goniometrics to be higher than 0.7, which statistically demonstrates the inter-rater and intra-rater reliability of goniometer measurements (Clapis, Davis, Davis, 2008; Engh, Fall, Hennig, & Soderlund, 2003; Nussbaumer, Leuing, Glatthorn, Stauffacher, Gerber, & Maffiuletti, 2010; Olson & Goehring, 2009).

Subjects completed an adapted version of the Occupational Questionnaire (Smith, Kielhofner, & Watts, 1986), after four hours once the equipment was removed. The subjects reflected on the occupations they completed during the four hours while wearing the equipment. This questionnaire allowed the researchers to understand the activities that the subjects participated in while wearing the instrument. The subjects categorized these activities, which allowed the researchers to examine the ranges of forearm rotation during these different activities. Subjects completed the first section of the Occupational Questionnaire, which required the subjects to categorize the activities they performed as work, daily living work, recreation, and rest. Work includes productive activities that are useful for other people which can be paid or voluntary (Smith, Kielhofner & Watts, 1986). Daily Living tasks are activities that are related to personal self care, such as housekeeping and shopping (Smith, et al., 1986). Rest is considered not doing anything in particular or taking a nap (Smith, et al., 1986). Any other activities that are not considered work, daily living, or rest are categorized as recreation. Subjects categorized these activities in 30 minute increments for the entire four hours that the accelerometers were worn. If the participants engaged in more than one activity during 30 minute time span, they listed all the activities and categorized each.

Researchers engaged the subjects in a semi-structured exit interview after wearing the instrument. This interview addressed any issues or concerns the subjects encountered while wearing the equipment and the subjects completed a likert scale assessing their comfort of the accelerometers and computer. Several open ended questions about the instruments impact on activity limitations and suggestions for future improvements were also included.

### Procedure

Subjects were scheduled for a four hour period during which they could wear the accelerometers. At this time informed consent was received from the subjects and the subjects completed a demographic form to determine if they met the inclusion criteria. Researchers measured the subjects ROM of elbow flexion and extension and forearm pronation and supination with a manual goniometer and recorded this on the demographic form.

Prior to attachment of the accelerometers to the subjects, accelerometers were calibrated using the following method. The accelerometers were positioned on an aluminum stand according to how they were placed on the subject. The metal stand represented the upper extremity in 90° of elbow flexion with the forearm in neutral position (0° of forearm rotation). The computer calibrated the accelerometers while they were on the stand. Researchers removed the accelerometers from the stand and placed them on the subject. One accelerometer was placed above the elbow, between the lateral and medial epicondyles of the humerus. The second accelerometer was attached on the dorsal surface of the forearm, over the distal aspects of the radius and ulna, avoiding the wrist joint. Subjects were instructed to position their arm in a standard starting configuration, with 90° elbow flexion and the forearm in neutral position, hands rotated so that the thumbs are pointing up. This starting configuration established the 0° reference angle for both forearm rotation and elbow flexion. The subjects were then asked to move through a full range of voluntary pronation and supination, allowing the system to record a baseline reading of the subjects' range of pronation and supination with the accelerometers. The computer was placed in a backpack and carried by the subject. This allowed the subject to leave the lab and complete their daily activities in their natural environment for four hours. After four hours, the subjects come back to the lab. Another baseline recording was taken of the subjects' range of pronation and supination. The researchers removed the accelerometers and equipment from the subjects and recorded ROM of the elbow and forearm using a manual goniometer as completed in the beginning of the study. Subjects completed an adapted version of the Occupational Questionnaire in reference to their time while wearing the accelerometers. Researchers also engaged the subjects in a semi-structured exit interview.

### Data Analysis

The computer software recorded the Euler angles (X, Y, and Z rotations) of both accelerometers. The accelerometers are set up in a parent child relationship, which allows data to be recorded on accelerometer's position and orientation in relationship to each other. The parent child relationship allows the computer software to collect data on the three Euler angles of the wrist sensor orientation with respect to the upper arm sensor. In this study, the Euler angles were used to describe the motion of the forearm and elbow. Euler X represents forearm pronation and supination, which is defined along the axis of the sensor aligned with the long axis of the forearm. Euler Y represents elbow adduction and abduction. The axis of Euler Y ‘floats’ between the two sensors as an intermediate step. Euler Z represents elbow flexion and extension, which is defined as rotation around the axis of the sensor aligned with the biomechanical axis of rotation of the elbow joint. The Euler angles were converted into degrees using a formula (Euler angle*180/π) in Microsoft® Excel.

Data was analyzed to determine if the software met the needs of the pilot study. More specifically, the data recorded in degrees was examined to determine if the software was capable of identifying the ranges of functional pronation and supination of the forearm. The frequency of use of forearm rotation was qualitatively examined. For this analysis the usage frequency was totaled for each 10° interval of rotation in order to evaluate rotation (pronation and supination), elbow flexion/extension, and elbow abduction/adduction for each subject. Then, the range of pronation and supination used by each subject was determined. Data more than three standard deviations away from the mean position of the forearm were considered noise and were not included in the analysis. For this study, the range of pronation or supination was defined as the ROM for which 95% of the recorded movements lay within. This range was calculated to the nearest 1° of rotation. The limits of pronation and supination for this range were then averaged across all subjects to determine the functional range of motion used for this subject pool for the types of activities undertaken.

The semi-structured exit interviews of all subjects were collectively analyzed to provide an overview of comfort and limitations of the equipment. Themes were developed based on common comments among the subjects in their semi-structured exit interview. The subjects' Occupational Questionnaires response were compiled together to present the percent of time that all subjects participated in the four main areas (work, daily living work, recreation, and rest) of occupation.

Reliability was evaluated by examining the initial and final baseline recording of pronation and supination for each subject subjectively. Inter-rater reliability was established by researchers following the same protocol to take goniometric measurements and set up subjects with the accelerometers. Reliability was also addressed by examining the data recorded on elbow adduction and abduction. Elbow adduction and abduction should remain fairly consistent because the elbow is a hinge joint that is not capable of performing this movement.

Validity was addressed by determining if the ranges of pronation and supination were consistent with literature on normal ROM for adults and the subjects' initial and final goniometric measurements. In order to address validity, data was analyzed to determine if there were any recordings that did not represent the ROM of the forearm accurately. If the computer software recorded ROM that was greater than the normal ROM of 90° for supination and 80° for pronation (Smith, Weiss, & Lehmkuhl, 1996), than this would be considered invalid data collection. Subject D was removed from the quantitative data analysis due to invalid data collection.

### Results

Accelerometers were used to collect data on subjects within their natural environment. The pilot instrument recorded data for approximately four hours while subjects completed a variety of daily activities including work, daily living work, recreation, and rest. 18 subjects participated in the study; the demographic information is represented in the following Table.

| Age: | |
|---|---|
| 18-21 | 13 |
| 22-25 | 5 |
| Gender | |
| Male | 2 |
| Female | 16 |
| Major | |
| OT | 15 |
| Engineer | 3 |
| Hand Dominance | |
| Left Hand | 0 |
| Right Hand | 17 |
| Ambidextrous | 1 |

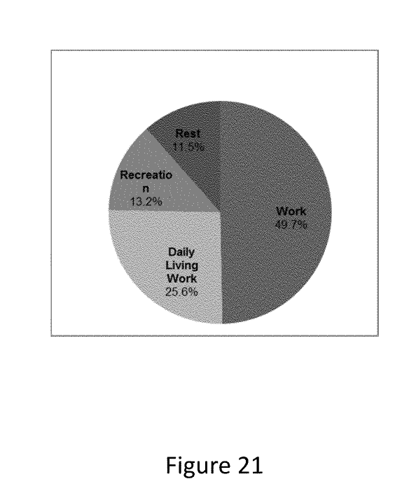

Data was collected from the Occupational Questionnaire and the results indicated that the 18 subjects participated in work related activities for 49.7%, daily living work for 25.6%, recreation for 13.2%, and rest for 11.5% of the time while they wore the instrument, refer to FIG. 21.

In addition to the demographic form, a semi-structured exit interview provided researchers with subjects' feedback about wearing the instrument. The subjects reflected back on their experience while wearing the instrument by answering several questions related to different aspects of the instrument which included pain, comfort level, and restriction of movement. The subjects reported an average of 1 out of 5 on a pain likert scale (1—no pain and 5—painful). The subjects reported an average of 2 out 5 on a comfort likert scale (1—comfortable and 5—extremely uncomfortable). A few of the subjects expressed that the computer on their back became warm over time.

Additional themes were developed based on the semi-structured exit interview and are represented in the following Table. According to the subjects' feedback, the accelerometers did not prevent them from completing any activities. Half of the subjects reported that they were inconvenienced by wearing the accelerometers. The subjects' comments consisted of the wires interfering with dressing, seating, and some daily living activities. The majority of the subjects indicated that they did not need to complete activities in a different way or adapt performance; however, some participants did make adaptations such as not wearing desired clothing. The majority of the subjects reported that the accelerometer straps kept the accelerometers in place during the entire duration of wearing the instrument except one subject reported tightening the accelerometers straps. Suggestions for improvement included more effective wire management, such as taping wires to the subjects, and the use of slimmer accelerometers.

Subjects' Feedback

| Wear of Instrument | Themes | Subjects' Quotes |
|---|---|---|
| Comfort | Straps were too | “The accelerometer became |
| | tight | tighter on my upper arm and the |
| | Computer was | computer started getting hot on |
| | warm on back | my back” |
| Pain | Caused no pain | “Not painful” |
| Restriction of | Dressing | ‘It was hard to put on a jacket |
| Movement | Seated position | because I had to adjust the |
| | | wires” |
| Inconvenience | Wire interference | “The wires got caught a couple |
| | Dressing | of times and sometimes I forgot |
| | Seated position | that I had the backpack on when |
| | Limited ROM | I sat down, so it got stuck when |
| | | I stood up” |
| | | “The backpack and wires |
| | | interfered with putting more |
| | | clothes on when cold” |
| Adaptation of | Driving position | “When I was doing my hair, I |
| Performance | Dressing | had to work around the wires” |
| | Self-care | |
| Ability of straps | Straps were loose | “There was one time I actually |
| to keep acceler- | | had to tighten the strap, but then |
| ometers in | | it stayed in place” |
| place | | |
| Suggestions | Wire management | “Tape wires to upper arm and |
| | | back” |

The computer software provided researchers with data that was used to determine the ranges and frequency of forearm rotation for each subject as well as the average for all subjects. Microsoft® Excel was used to calculate and graphically represent the subjects' ranges and frequency of forearm rotation. According to the analyzed data, the average range of pronation and supination for the subjects was 44° of pronation with the standard deviation of 14° and 71° of supination with the standard deviation of 15°, refer to the Table below for specific information.

Functional Ranges of Pronation and Supination of Subjects

| | | | | | | | | | | | | | | | | | | | Standard |
|---|---|---|---|---|---|---|---|---|---|---|---|---|---|---|---|---|---|---|---|
| Subject | A | B | C | E | F | G | H | I | J | K | L | M | N | O | P | Q | R | Average | Deviation |
| Pronation | 55 | 50 | 37 | 25 | 54 | 46 | 73 | 51 | 35 | 46 | 53 | 53 | 8 | 39 | 49 | 38 | 40 | 44 | 14 |
| Supination | 75 | 67 | 75 | 88 | 45 | 73 | 64 | 45 | 67 | 57 | 102 | 73 | 80 | 64 | 63 | 72 | 92 | 71 | 15 |

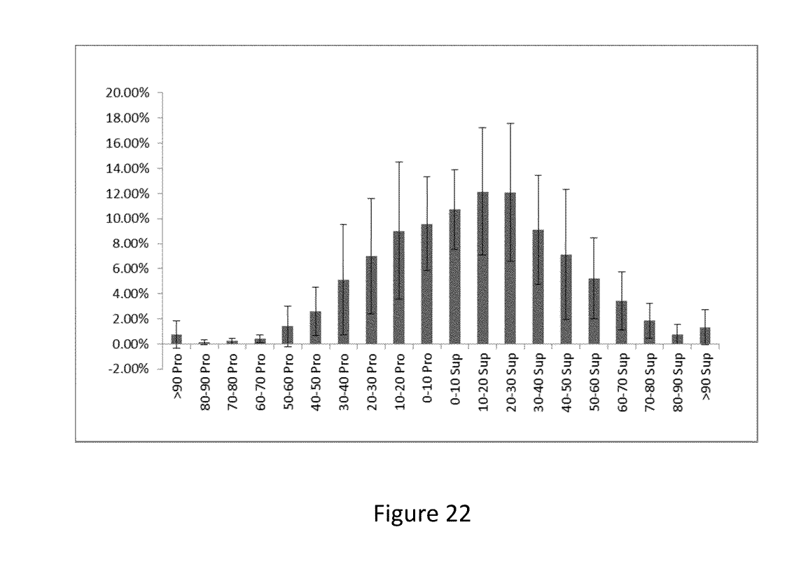

FIG. 22 is a histogram that represents the average percent of time that all subjects were in each interval of pronation and supination. The error bars reflect the variability of range of motion between subjects. The range varies for each subject because they were not completing the same tasks. There was a wide range of tasks completed while wearing the equipment which included eating, taking notes, computer use, reading, and self-care. The subjects were in supination more frequently than pronation and needed a greater range of motion for supination for the tasks that the subjects completed.

Reliability and validity of the instrument was addressed through evaluating the quantitative data. The base line recordings were subjectively identified as being within the normal range of motion for pronation and supination based on the subjects' initial and final goniometric measurements as well as referring to the normal ROM of 90° for supination and 80° for pronation (Smith, et al., 1996). The ROM for elbow adduction and abduction for each subject remained consistent with little variation of movement during the four hours while they wore the equipment. The ranges of pronation and supination for the subjects were consistent with the normal ROM for adults, reported by Smith, et al. (1996), as well as each subjects' initial and final goniometric recordings. Any recordings that were greater than 80° pronation and 90° supination (Smith, et al., 1996) were considered invalid data. There were minimal recordings outside the normal range of 80° pronation and 90° supination (Smith, et al., 1996), suggesting there was minimal invalid data.

### Discussion

Due to the ambulatory nature of the pilot instrument, data for this study was able to be collected in the subjects' natural environment. The ability to use the instrument in the subjects' natural environment allowed researchers to gain a more realistic representation of functional forearm rotation than if completed in a controlled environment. By collecting data in the natural environment, researchers were able to evaluate a range of daily activities that the subjects chose to complete rather than specific activities chosen by the researcher. Previous studies examining functional forearm rotation were completed in a controlled environment with specific activities performed. However, research has found that there is a difference in subjects' performance between the laboratory and the subjects' natural environment (Coley et al., 2008), which supports the need for studies to be completed in the subjects' natural environment.

The functional ROM of forearm rotation determined from this research is not consistent with previous research completed on forearm rotation. The ROM determined by this study for supination was 71° with the standard deviation of 15°, which is much greater than the previously determined range of 50° of supination by Morey et al. (1981). The ROM determined by this study for pronation was 44° with the standard deviation of 14°, which is relatively similar to the previous finding of 50° of pronation (Morey et al., 1981). Morey et al. reported standard deviations for the ROM used during each specific position and task completed. The standard deviations of the various positions and tasks ranged from 16° to 23.8°. The difference in the reported findings of functional ROM for the forearm could be influenced by several factors. The variation may be due to the different method for collecting data on functional forearm rotation. The study completed by Morey et al. was conducted in a laboratory environment with a set of specific ADLs and positions needed to complete personal care and hygiene. Several of the activities that were used during the study completed by Morey et al. included meal preparation, feeding, reading, and using the telephone. The difference in how subjects' complete tasks in a natural vs. controlled environment (Coley et al., 2008), could influence the ROM subjects use to complete activities.

The range of pronation and supination determined from this pilot study represents activities that the subjects completed while wearing the instrument. The exact activities completed during the four hours were not determined, although the subjects provided brief examples of some activities they completed within the four main areas of occupation, according to The Occupational Questionnaire. The subjects reported completing activities in the following four main areas of work, daily living work, recreation, and rest. The recorded ROM from this study was larger for supination, which could have been influenced by the activities the subjects reported completing as college students. The subjects reported spending almost half of their time in class, computer labs, or completing school work. One of the four main categories from The Occupational Questionnaire, daily living work, was minimally represented due to the report from subjects that they spent their time eating rather than completing laundry, meal preparation, and self-care. Therefore, the range of forearm rotation may not be an accurate representation of the four major areas of occupation. The data collected from the 17 subjects may be suggestive of the functional forearm rotation used by college students to perform college related activities. However, further studies would need to be completed to address the ROM needed to perform activities related to all areas of occupation.

Another factor that could contribute to the difference in the range of supination could be the increasing use of technology. Technology use has increased since the study by Morey et al. (1981). Individuals are completing new activities involving computers, cell phones, and MP3 players. As these activities become more frequently expected and completed, in daily routines, additional research is needed to further assess ROM requirements for these particular activities.

### Application to UE Rehabilitation

Once the instrument is conducted on a larger and more diverse population, the data collected can be used to build a normative knowledge base on functional forearm rotation. The normative information on UE biomechanics has the ability to enhance clinical practice by helping professionals evaluate, diagnose, and treat UE pathologies (Bryce & Armstrong, 2008; Zimmerman, 2002). ROM is one of the areas that occupational therapists address in the clinic. In order to address ROM, it is necessary for therapists to have knowledge of normal ROM required to perform daily activities. With baseline knowledge of functional ROM, therapists will have the knowledge to treat UE conditions and pathologies (Bryce & Armstrong, 2008; Zimmerman, 2002). Different surgeries and pathologies of the UE affect ROM, leading to the focus of occupational therapy to be regaining functional movement. The pilot instrument used in this study would provide the surgeons and clinicians with a standard baseline of forearm rotation to follow when addressing ROM of the UE for a wide range of pathologies and surgeries.

In addition, medical and adaptive devices can be enhanced by the knowledge of functional ROM that is provided by this instrument. The development of medical and adaptive devices can incorporate the data of normal movement to create more effective devices that can enable completion of daily activities, such as an adaptive spoon that addresses adaptation of forearm rotation in order to complete feeding. Knowledge of normal body movement of the forearm and wrist can also aide in the development of a new replacement wrist joint due to the need of knowing the normal ROM used by the wrist.

The instrument allows data to be collected on simultaneous movement, which can be used to evaluate other UE movement such as elbow flexion and extension, wrist flexion and extension, and ulnar and radial deviation.

Not only does the instrument have the potential to be used to contribute to the normative data of UE movement, but the possibility of using the instrument in the clinic for intervention. The instrument demonstrates the ability to capture real-time motion which allows analysis of the range of movement used within a particular activity. Additionally, the instrument would allow evaluation of the quality of movement in reference to evaluating motor performance in orthopedic and neurological populations. Individuals with orthopedic conditions such as tendon transfers and individuals with neurological conditions, such as stroke, may need to relearn motor patterns. The instrument used in this study may be able to assist in the relearning of motor patterns. The instrument could provide the clinicians and patients with concrete and immediate feedback on the range and flow of their UE movement. The analysis of the quality and range of movement can be used to provide clinicians and patients with a better understanding of their performance and limitations.

### Example #3

The purpose of the E-MAT is to produce an affordable, lightweight tool that can be used in the clinic with neuromuscular re-education. This study looked at motor control theory, which is the ability to regulate or direct the mechanisms essential to movement (Shumway-Cook & Woollacott, 2001) as well as real-time motion capture which applies motion-sensing technology to produce highly accurate information to describe performance (Lehrer, Attygalle, Wolf & Rikakis, 2011). The purpose of this study was to further develop Real-Time Motion Capture Technology utilizing the Electronic Movement Analysis Tool (E-MAT) in neuromuscular re-education, assessing the utility from a clinical perspective. From this study, the E-MAT's current measurement tool and computer application can be further developed for clinical assessment of motor control in rehabilitation.

For the purpose of this study the E-MAT will be compared to the Motor Assessment Scale (MAS). The MAS was designed to quantitatively measure motor recovery of stroke victims using functional tasks (Asher, 2007). The MAS consists of eight movements scored on a 7-point ordinal scale ranging from 0 to 6. A score of 6 indicates optimal behavior. The MAS has been successfully used as an outcome measure in published efficacy studies of clinician intervention. (Radomski & Trombly, 2008). The MAS was chosen because the researchers wanted to compare the E-MAT to a standardized assessment that was currently used in the clinic. The MAS was appropriate because it uses functional tasks and is used with stroke victims.

### Participants

This study used a methodological research design through a descriptive approach. Purposive sampling elicited two participants from a local outpatient neurological rehabilitation clinic. The researchers recruited an occupational therapist and a client from the therapist's current caseload. Inclusion criteria for the therapist included: a licensed occupational therapist, must currently treat individuals with a motor control deficit, and must be able to refer a client for this study. The therapist must also give consent to be a part of the study and English must be a primary language. Inclusion criteria for the client: must be seeking occupational therapy services for improved motor control, must speak English as their primary language, and may not be diagnosed with any other neurological/orthopedic conditions that could interfere with data collection. Client must also give consent to participate.

### Instruments

MotionNodes.

MotionNodes are small, light and low-cost instruments that collect major motions of joints (Bakke & Tokheim, 2010). MotionNodes are instruments that monitor movement with minimal interference and are manageable in normal environments (DeGoede et al., 2011). The MotionNodes measure acceleration along three anatomical axes, (X, Y, and Z rotations) with respect to the earth (DeGoede et al., 2011). Two MotionNodes can be set up in a parent-child relationship for data to be recorded on two joint positions and orientation to each other. Research shows that placement is very critical to maintain reliability of the tool environments (DeGoede et al., 2011). The prototype of the E-MAT identified proper MotionNode placement for elbow measurements as follows: One is placed above the elbow, between the lateral and medial epicondyle of the humerus and the second is attached on the dorsal surface of the forearm, over the distal aspect of the radius and ulna. In addition to MotionNode placement, the arm must be positioned in ninety degrees of elbow flexion, with the forearm in neutral position so that the hand is rotated with the thumb pointed up (DeGoede et al., 2011).

In this study, the MotionNodes were attached by Moleskin between the forearm placement and the MotionNode. Tubigrip was worn on top of each MotionNode to ensure secure attachment and to help with wire management during movement. During this study, two MotionNodes were attached to the forearm to measure joint movement of the wrist relative to the elbow, specifically flexion and extension, and pronation and supination as similarly shown in FIG. 2.

Computer Software.

MotionNodes connect to any computer via USB port. The computer software provides data by measuring the relative position of one MotionNode to the other. For this particular study highlighting the elbow joint and forearm rotation, two graphs were displayed demonstrating flexion and extension of the elbow and pronation and supination of the forearm in motion. In conjunction with the MotionNodes, the computer software applies a real-time motion capture system to create a movement trace.

The computer software can collect data from the MotionNodes and graph movements in real-time. The computer software is composed of two systems, the E-TRK and the E-LOG. The E-TRK graphs movement in real-time and then allows for the emulation of the same movement, as shown similarly in FIG. 3. The E-TRK immediately displays the emulation of movement on a graph. The E-LOG allows users to save the graph's data points into an Excel document for measuring progress of the targeted extremity over time.

### Outcome Measures

Semi-Structured Interview.

The student researcher created two semi-structured interviews one directed to the therapist and one directed to the client. The interviews were conducted after the therapist and client had been exposed to the E-MAT in the therapy session. The interview questions facilitated a discussion about the clinical utility of the E-MAT.

Motor Assessment Scale (MAS).

The Motor Assessment Scale (MAS) is a performance-based scale that was developed as a means of assessing everyday motor function in patients with stroke (Carr, Shepherd, Nordholm, & Lynne, 1985). The MAS is based on a task-oriented approach to evaluation that assesses performance of functional tasks rather than isolated patterns of movement (Malouin, Pichard, Bonneau, Durand, & Corriveau, 1994). The researchers wanted to expose the therapist to an assessment that is currently used in the clinic and compare the E-MAT and the MAS in the semi-structured interview.

### Procedures

An Institutional Review Board (IRB) gave approval to the researchers to conduct this study. Informed consent was provided by the therapist and client. The therapist was contacted through email and phone conversations and the therapist suggested a client that the therapist was currently treating that the therapist felt was appropriate for the study.

For the actual research and intervention session, the student researcher set up the E-MAT computer software. The computer was placed in front of the client so the graphs were visible to the client and therapist while the therapy session took place. The student researcher then placed the MotionNodes on the client. The client performed three different tasks while wearing the E-MAT. First the therapist guided the client through the movement of stacking cones to create the real-time movement pattern. Then the client tried to emulate the same motion without the help of the therapist. Second the therapist guided the client through the functional task of folding a towel to form a new pattern. The client then tried to emulate the motion. And finally the therapist guided the client through the activity of stacking cones this time with the metronome feature of the E-MAT. Again, this was followed by having the client emulate the graphed motion.

After the client and therapist were exposed to the E-MAT the student researcher performed the MAS in part. Sections 6 and 7 were performed of the MAS. This focused on upper arm function and hand movement, which were relative to the client and the E-MAT. The semi-structure interview for the client was then performed followed by the semi-structured interview with the therapist.

### Data Analysis

The researcher took the responses from the semi-structured interview, categorized the responses and put them in a chart format. The researcher then found common responses that emphasized themes as the therapist and client felt the E-MAT had clinical utility.

Data collection was completed during one therapy session that lasted one hour. One therapist and one client participated in data collection.

### Participants

Participant 1 (therapist): A licensed occupational therapist has been practicing for 10 years who has been working at the current neurorehabilitation outpatient clinic for 4½ years. The therapist first worked as a certified occupational therapist assistant before becoming an occupational therapist. The therapist has also worked in numerous settings such as acute care, long-term care, inpatient rehabilitation, and outpatient therapy.

Participant 2 (client): A 71-year-old male who suffered from a cardiovascular accident in January of 2013. Symptoms of the CVA include left sided weakness, poor control of left upper extremity, and Apraxia. Participant is right hand dominant. His past medical history consists of Lumbar surgery, degenerative joint disease, chronic sinusitis, cellulites due to a dog bite, and hypertension. The participant was independent before the CVA and worked full time at a hotel in the laundromat. The participant was working with the occupational therapist on Activities of Daily Living (ADL's), fine motor skills, gross motor skills and functional tasks to help him return to work. The therapist stated that the client has come to past session wearing his clothes inside out and not being aware of it. Goals included increase fine motor in hand to increase score on 9 hole peg test, increase bilateral strength for folding laundry, increase gross motor and increase left hand digit strength to complete ADL's.

### Therapy Session Described

The student researcher started the session by presenting the purpose of the study and going over the consent form with the participants. To start the procedures for the study, the MotionNodes were placed on the elbow between the lateral and medial epicondyle of the humerus and the second node was attached on the dorsal surface of the forearm in neutral position. The computer was placed in front of the client and the therapist so the screen and computer program was visible. The tasks that were completed during the session were chosen by the therapist. She chose stacking cones and folding a towel. The therapist and client responded well to the setup of the E-MAT and wanted to move forward with the session. For each task the movement was graphed once and then emulated 2 to 3 times. The movement was graphed for 30 seconds. Two graphs were present as the movement was graphed and emulated; one for flexion and extension of the elbow and one for supination and pronation of the forearm. The emulation of the movement was performed directly right after the movement was graphed.

First the therapist guided the client using a hand-over-hand technique through the movement of stacking cones to create the real-time movement pattern. Then the client tried to emulate the same motion without the help of the therapist. The movement captured by the E-MAT was primarily addressing flexion and extension of the elbow. During this task the client was able to watch the real-time movement of the graph while emulating the motion. The client switched between looking at the graph while he emulated the motion and watching the task. The client was able to successfully stack the cones however, the movement he emulated did not fully match the graphed movement because the cones were not placed in the same spot and the client did not understand that the cones were only to be stacked two cones high as he was guided to do. When he emulated the motion himself he stacked the cones three cones high changing the movement of elbow extension.

Second the therapist guided the client through the functional task of folding a towel to form a new pattern. The therapist had him fold the towel in half, and then in half again. The client then tried to emulate the motion. This task was focusing on both flexion and extension of the elbow and pronation and supination of the forearm. During the emulation of the movement the client was not focused on the graph as he was focused on the task. During the completion of the task the towel became folded and the client attempted to fix the fold. This was not part of the graphed movement so the emulated movement went outside of the graph. The client was unsuccessful in completing the task of folding the towel in the same way he was guided to do so. When the client looked at the graph after the allotted time of 30 seconds was completed he stated, “I did it wrong.”

And finally the therapist guided the client through the activity of stacking cones this time with the metronome feature of the E-MAT. This was the same movement as the first task however the client was instructed to only listen to the noise and pay attention to the task and not the graphs. When the client went to emulate the motion the client had difficulty understanding the purpose of the metronome. The client was to pick up a cone with one click and put it down with the second click, instead the client tried to stack the cones as fast as he could. After the third trial, the therapist and the client stated that they felt that the metronome sounded like a timer so the client was trying to rush through the task. This resulted in the emulated movement not being close to the graphed movement but the client was able to stack the cones.

While the client completed the three tasks the therapist watched both the client and the graphs to see how well the client was emulating the movement. The therapist was also looking to see how the E-MAT was measuring the smoothness of movement that the client was completing.

After the three trials of the E-MAT the student researcher performed the MAS in part. Sections 6, Upper Arm Function and 7, Hand Movements, were performed of the MAS. Section 6 was only performed in part because only relative tasks were completed. The movements from section 6 that were completed were; sitting hold extended arm in forward flexion at 90 degrees to body for 2 seconds, and sitting client lifts arm to above position, hold for 10 seconds and then lowers. In section 7 all tasks were completed. This included extension of the wrist by lifting a cylindrical object in the palm of the client's hand, radial deviation of the wrist, pronation and supination of the wrist with elbow unsupported in a right angle, sitting the client reached for a large ball with both hands lifted it up and put it back down, sitting the client picked up a polystyrene cup from the table and put it across the other side of his body, and sitting continuous opposition of thumb and each finger. The client was able to preform all tasks from the MAS with the exception of the last task in section 7. The client was able to perform opposition of the thumb to each finger however he was not able to do it 14 times in 10 seconds. The tasks that were performed with one hand were performed on the client's affected side. After the participants were exposed to the E-MAT in the therapy session the participants and the student researcher completed the semi-structured interview.

### Qualitative Data

The purpose of the semi-structured interview was to receive feedback from the therapist and client. The following table shows the main questions from the semi-structured interview that the researchers were interested in and the summarized responses from the participants. The therapist and client were not asked the same questions because some of the questions did not pertain to the client. The questions were designed to see how the therapist and client enjoyed using the E-MAT, if it accurately measured smoothness of movement, if the therapist and client felt the E-MAT could be used in future interventions and if the participants had any suggestions for the future development of the E-MAT.

| Semi-structured Interview | Summarized Therapist | Summarized Client |
|---|---|---|
| Questions | Response | Response |
| How accurate do you feel the | The E-MAT was proficient in | Felt that the E-MAT showed |
| software was in measuring | recording smoothness of | him how coordinated he was. |
| smoothness of movement? | movement and the visual | |
| | feedback was utilized when | |
| | emulating movement patterns | |
| | of the upper extremity. | |
| | The visual feedback during | |
| | functional tasks was used | |
| | more by the therapist to | |
| | observe the emulation of | |
| | movement but the client only | |
| | focused on the task. | |
| How did the information from | Not familiar with the MAS. Felt | N/A |
| the software compare to the | that the MAS was looking at | |
| information given by the MAS? | movement but liked that the E- | |
| | MAT could be used with a | |
| | functional task and the MAS | |
| | could not. | |
| Do you use any objective | No. Uses the 9 Hole Peg Test | N/A |
| assessments to measure | and Manual Muscle Testing | |
| motor control with your | | |
| client's? | | |
| How could the E-MAT be used | The therapist would start with | The therapist could look at the |
| to plan an intervention | a bigger “river” so the client | score from the E-MAT and see |
| session? | has a better chance of getting | the progress over time. |
| | a higher score and then | |
| | gradually make the “river” | |
| | smaller in future therapy | |
| | sessions to show progress. | |
| What are some of the overall | Easy to use, lightweight, and | The E-MAT could be used in |
| benefits of the E-MAT? | the visual feedback was | his intervention session to help |
| | beneficial. | him gain his strength back. |

The main points of the interview were that the therapist felt the E-MAT measured smoothness of movement accurately, that the client and therapist felt the E-MAT could be used in an intervention session, and that the E-AMT is easy to use, lightweight, and the visual feedback was beneficial. The client and therapist commented on the ease of use of the E-MAT but had suggestions for visual display changes. The critiques of the E-MAT were beneficial. One critique that was not mentioned from the therapist but was noticed by the student researcher was that the client was able to complete the tasks but that the client did not receive a high score from the E-MAT.

### Application to Occupational Therapy

As per our participants, this study demonstrated that the E-MAT has good clinical utility. The feedback provided by the therapist suggested that the E-MAT measures smoothness of movement and can be used in an intervention session to track progress. The researches of this study were looking at the E-MAT specifically to motor control theory and motor learning.

The therapist commented that the visual feedback, a unique aspect of the E-MAT was a benefit. Specifically, real-time motion capture technology used in the E-MAT gives the client extrinsic feedback about his/her movement patterns. Repeating movement patterns during intervention tasks provides a patient with memory on the outcome of the movement; this is called knowledge of results (KR) (Shumway-Cook & Woollacott, 2001). Extrinsic feedback can be provided through KR as the movement's goal is remembered and repeated. However, knowledge of performance (KP) is also an applicable extrinsic feedback approach to the E-MAT's application (DeGoede et al., 2012). For instance, with the E-MAT, as the movements are performed by the client, KP feedback is provided to the client about the movement pattern utilized to achieve the goal (Shumway-Cook & Woollacott, 2001).

As stated in the literature repeating movement is beneficial to recovery especially in distributed practice. The E-MAT can be used in intervention sessions using distributed practice because the therapist is able to customize the practice time and the rest time and the E-MAT is capable of storing and producing applicable graphs throughout these time periods. Thus, the unique features of the E-MAT make it applicable in the clinic with clients with motor control deficits.

The results of this study supports, and is also suggested from the therapist's feedback, that the E-MAT could be used for functional tasks.

### Example #4

Motor control theory states that all motor acts result from an interaction of the person, the environment, and the task (Holt, 2005). Tremor is defined as an “involuntary, rhythmic oscillation of a body part” (Alty & Kempster, 2011, p. 623). Tremor negatively impacts motor control by altering the capabilities of the person. Additionally, past research illustrated that tremor negatively impacts participation in daily activities (Politis et al., 2010). Because there is no cure for tremor in and of itself, motor control rehabilitation typically utilizes compensatory strategies, such as adapting the environment or the task, to allow for motor control that is adequate for successful participation in daily life (Clarke et al., 2009; Forwell, Copperman, & Hugos, 2008; Gage & Storey, 2004; Heisters, 2010; Melnick, 2001). In current rehabilitation, clinicians assess tremor severity primarily through clinical observation (Alty & Kempster, 2011; Bajaj et al., 2010). Relying solely on observation is problematic because observation is not objective and provides no means by which to quantify patient responses to compensatory intervention approaches over time (Alty & Kempster, 2011; Bajaj et al., 2010). Therefore, developing objective biomechanical instruments capable of measuring tremor in clinical settings is important to document the effectiveness of motor control rehabilitation on managing tremor.

### Motor Control Theory

Motor control theory states that all motor acts result from the interaction of the abilities of a specific person, the demands of the environment, and the demands of the task (Holt, 2005; Mastos, Miller, Eliasson, & Imms, 2007). Barriers in any of these dimensions negatively affect motor control, resulting in difficulties engaging in daily activities (Holt, 2005; Mastos et al., 2007). Whenever motor control deficits are present, motor control rehabilitation is necessary to remediate or compensate for specific barriers, in order to again allow for motor control that is adequate for engagement in necessary and meaningful daily activities. Rehabilitation specialists couple motor control theory with goal-directed training; rehabilitation focuses on incorporating the use of specific movements to obtain a functional goal that is meaningful to the patient (Mastos et al., 2007).

Therapy focuses either on remediating underlying components of motor control or on adapting the environment or task to compensate for deficits in motor control (Forwell et al., 2008; Mastos et al., 2007). It is important that motor control rehabilitation specialists use objective manners to measure the effectiveness of interventions to provide evidence-based care, as well as to guarantee insurance reimbursement for therapy services (Holt, 2005). To ensure objective measurement of therapy outcomes and patient progress, biomechanical instruments, based in motor control theory, are developed (Holt, 2005). Such instruments can be used to document the effectiveness of remedial therapy as well as functional gains from compensatory therapy (Holt, 2005). Biomechanical instruments can be used with individuals who have a variety of medical conditions that negatively influence motor control. The present inquiry will focus on tremor, which is a specific symptom that negatively affects motor control by altering the capabilities of the person.

### Tremor

Tremor, the most prevalent movement disorder in clinical medicine, is defined as an “involuntary, rhythmic oscillation of a body part” (Alty & Kempster, 2011, p. 623). Different types of tremor exist and are often indicative of specific medical conditions. Causes of tremor can range from association with a neurological condition, such as Parkinson's Disease (PD), to the use or abuse of prescription and non-prescription drugs (Alty & Kempster, 2011; Damjanov, 2012). Despite the cause, the frequency of tremor is measured scientifically using surface electromyography, and it is reported in hertz (Hz). The most common forms of tremor range from 4-12 Hz (Puschmann & Wszolek, 2011). Amplitude of tremor is assessed primarily through clinical observation and is classified as low, moderate, or high (Puschmann & Wszolek, 2011). Two main categories of tremor exist, and consist of action tremor and rest tremor. Action tremor occurs with voluntary contraction of muscles and can present under a variety of conditions. Rest tremor occurs when muscles are completely supported against gravity and are not under voluntary contraction. Rest tremor will slow down or stop with limb movement (Alty & Kempster, 2011; National Tremor Association, 2012; Puschmann & Wszolek, 2011).

Because there is no cure for tremor in and of itself, motor control rehabilitation typically utilizes compensatory strategies, such as adapting the environment or the task, to allow for motor control that is adequate for successful participation in daily life (Clarke et al., 2009; Forwell et al., 2008; Gage & Storey, 2004; Heisters, 2010; Melnick, 2001). Due to a strong association between PD and tremor, there is a prevalence of literature that focuses on examining how tremor associated with PD presents and is measured. When considering clients who experience tremor as a result of conditions other than PD, the research about PD and tremor is still helpful to consider.

Contradictory evidence exists when describing the impact that tremor has on participation in daily life. Melnick (2001) reported that tremor is a cosmetic issue that rarely interferes with activities of daily living. Hariz and Forsgren (2011) found that those with PD dominated by postural instability and gait difficulties experienced more objective difficulties in activities of daily living than did those with tremor. In opposition to these findings, Politis, et al. (2010) conducted a small qualitative study in which patients with PD identified tremor as the only motor symptom that negatively impacted participation in daily life in both the early and late stages of the disease. Clearly, variability exists within the medical community as to how tremor impacts participation in daily life. The nature of the research presented may explain some of this variability. For example, while Melnick (2001) synthesized available literature relative to the impact of tremor on participation in daily life, Hariz and Forsgren (2011) compared two different sub-groups of individuals with PD using objective rating scales, and Politis, et al. (2010) obtained subjective views of a group of individuals with PD at different stages of the disease process. While none of these results can be generalized to all persons who experience tremor, it is important for motor control rehabilitation specialists to understand the importance of addressing tremor in therapy whenever it interferes with participation in daily life.

### Clinical Assessment of Tremor

While scientists use surface electromyography to measure tremor in laboratory settings, clinicians predominately rely on observation to determine the severity of tremor in real-life settings (Alty & Kempster, 2011; Bajaj et al., 2010). The Unified Parkinson's Disease Rating Scale (UPDRS), motor examination subscale is often the means by which clinicians structure and rate their observations of tremor in patients with and without PD, as the UPDRS is one of the only tools available to healthcare providers that aids in structuring clinical observations of tremor (Alty & Kempster, 2011; Bajaj et al., 2010). Relying solely on observation is problematic because observation provides no means by which to quantify tremor in an objective manner that is consistent across raters (Alty & Kempster, 2011; Bajaj et al., 2010). Thus, it is impossible to determine if clinical observation is truly accurate. Many researchers identified and addressed the current lack of objective clinical measures of tremor (Bajaj et al., 2010; Caligiuri & Tripp, 2004; DeGoede, Panchik, Paranto, Seymour, & Speiden, 2012; Politis et al., 2010; Rigas et al., 2012; Salarian et al., 2007).

### Objective Measurement Tools

Wearable Sensors.

Wearable sensors provide a cost-effective manner with which professionals can obtain accurate and objective measures of specific movements while a client engages in functional tasks in a variety of settings. Therefore, wearable sensors possess clinical utility in diagnosing tremor, as well as in measuring the effectiveness of interventions on managing tremor severity (Bonato, 2005).

Biomechanical Assessments of Tremor.

The existing literature relative to the development of biomechanical instruments used to measure tremor tends to neglect the clinical utility of such tools, as such instruments were developed and tested in controlled laboratory settings; therefore, measures of their effectiveness cannot be generalized to real-life, clinical settings (Caligiuri & Tripp, 2004; Rigas et al., 2012; Salarian et al., 2007). Additionally, instrument development thus far focused on aiding in the diagnosis and pharmacological management of tremor, particularly in patients with PD (Caligiuri & Tripp, 2004; Rigas et al., 2012; Salarian et al., 2007). While it is important to address diagnosis and pharmacological management of tremor, it is also necessary for motor control rehabilitation specialists to use objective manners to document the effectiveness of their interventions, as these specialists play a critical role in managing tremor severity. However, a gap in the current literature exists in terms of using objective biomechanical instruments with documented clinical utility to measure the impact of motor control interventions on tremor management.

The purpose of the current study was to examine the clinical utility of the E-MAT in measuring the effectiveness of compensatory occupational therapy interventions for tremor suppression. The current study utilized a case study format to gain an in-depth understanding of how one occupational therapist and one patient who experiences tremor view the clinical utility of the EMAT in objectively measuring tremor severity as an aide to examining functional outcomes gained through compensatory therapy. The specific research questions of the current study are as follows: How does a practicing occupational therapist view the clinical utility of the E-MAT in measuring the effects of compensatory intervention in suppressing tremor? How does a patient who experiences tremor view the clinical utility of the E-MAT in measuring tremor suppression? What are the views of the therapist and patient on improving the design of the E-MAT for future use?

### Design

The current study utilized a methodological research design to gain preliminary data relative to the clinical utility of the E-MAT in measuring tremor. A descriptive case study design was utilized to obtain information relative to the future development of the tremor application of the E-MAT software. A case study format enhanced the methodological design of the current study by directing the initial pilot test of the software with the specific population for which it was designed.

### Participants

Purposive sampling was used to recruit applicable participants for the current study. The researchers recruited one occupational therapist from a local outpatient neurological rehabilitation clinic via phone and e-mail correspondence. Inclusion criteria for the therapist were: the individual must be a licensed occupational therapist, the therapist must treat individuals who experience tremor, and the therapist must have access to a patient whom they can refer to this study. From the therapist's case load, one patient was identified and recruited to participate in this study. Inclusion criteria for the patient were: the patient must experience tremor that negatively impacts participation in daily activities, the patient must be seeking outpatient occupational therapy services to compensate for tremor and improve motor control, and the patient must speak fluent English. Ethical approval was obtained from the Institutional Review Board of the participating comprehensive college as well as from the Institutional Review Board of the outpatient neurological rehabilitation clinic. Written informed consent was also obtained from both participants prior to data collection.

### Instruments

E-MAT. The E-MAT consists of two MotionNodes and a computer program written with software specific to those MotionNodes. The E-MAT system can be used to measure smoothness of movement or to measure the acceleration of a body part. For the purpose of the current study, the acceleration application, used to measure tremor, is the focus of discussion. The computer software was written to collect objective data about the acceleration of a body part by specifically analyzing the frequency and amplitude of that movement. The present study tests the tremor application of the E-MAT software in a clinical setting.

MotionNodes.

MotionNodes are small, lightweight, and low-cost instruments that collect objective information about the motions of joints (Bakke & Tokheim, 2010). MotionNodes consist of an integrated accelerometer, gyroscope, and magnetometer. Accelerometers provide information about acceleration and the angular rate of body movement in real time, while gyroscopes measure rotation of a body segment, and magnetometers measure orientation of a body segment in relation to the magnetic field of Earth (Bakke & Tokheim, 2010). MotionNodes measure acceleration along three anatomical axes (X, Y, and Z), with respect to the position of Earth in its rotation (Bakke & Tokheim, 2010). MotionNodes monitor movement with minimal interference from outside sources, such as radiofrequencies (Bakke & Tokheim, 2010).

The E-MAT is equipped with two MotionNodes that are capable of measuring the motion of the first node relative to the static position of the second node. Because the focus of the present inquiry was on measuring acceleration of a specific body segment, namely tremor of the hand, it was only necessary to use one MotionNode. One MotionNode was placed on the dorsal surface of the patient's forearm, over the distal aspect of the radius and ulna. Moleskin, placed on the back of the MotionNode, attached the MotionNode to the skin, and Tubigrip, worn around the outside of the MotionNode, secured the attachment of the MotionNode to the patient's skin.

Computer Software.

The MotionNode was connected to a windows computer via a USB port. The computer software, written specifically for the E-MAT, is comprised of an E-LOG system and an E-TRK system. The student researcher utilized the E-TRK system to collect acceleration data, which reported a numeric representation of the frequency and amplitude of tremor, as the patient participated in functional therapy activities with and without compensatory strategies to manage tremor.

The Unified Parkinson's Disease Rating Scale (UPDRS).

The student researcher also introduced the UPDRS, motor subscale to the occupational therapist to determine her opinion as to how the measures made by the E-MAT compared to those made by the UPDRS. Asking the therapist to compare the two tools enhances the quality of the information obtained through the current study. Although no clinical “gold standard” measure of tremor currently exists, the UPDRS is widely used by a variety of healthcare professionals to assess tremor severity in patients with and without PD (Alty & Kempster, 2011; Bajaj et al., 2010), so it is important to understand how the occupational therapist rates the measures made by the E-MAT relative to those made based on the UPDRS.

### Outcome Measures

Semi-Structured Interviews.

Two semi-structured interviews were used, one specific to the occupational therapist, and another specific to the patient. The interviews were conducted after exposing both parties to the E-MAT during one typical therapy session. The therapy session was planned by the occupational therapist in accordance with the patient's plan of care. The semi-structured interviews were designed to obtain the opinions of the therapist and the patient relative to the clinical utility of the E-MAT, as well as their views on how to improve the tool for future clinical use.

### Procedure

Prior to the initiation of the current study, the researchers obtained ethical approval from the Institutional Review Board of the participating comprehensive college and the Institutional Review Board of the participating outpatient neurological rehabilitation clinic. Both participants signed an informed consent document before participating in the present study. The informed consent document explained that the therapy session would be conducted by the occupational therapist according to the patient's plan of care, and that the session would be billed to the patient's insurance company in the same manner as any other therapy session.

Two semi-structured interviews were designed to conduct with the participants after exposing them to the E-MAT tremor software. The student researcher then attended one therapy session. During the therapy session, the student researcher attached one MotionNode to the patient using the aforementioned placement and attachment methods. The tremor software of the E-MAT was used to take measurements of the frequency and amplitude of the patient's tremor during compensatory therapy activities, as prescribed by the occupational therapist according to the patient's plan of care. The student researcher initially took E-MAT readings during functional activities without any type of intervention to provide a baseline measure of tremor severity. The student researcher then took reading while the patient used a wrist cock-up splint, arm weights, and weighted utensils during functional activities. The student researcher shared the measurements made by the E-MAT with the therapist and the patient throughout the session. After the session, the student researcher conducted one semi-structured interview with the occupational therapist and one semi-structured interview with the patient.

### Data Analysis

The student researcher took field notes during the semi-structured interviews. The researchers then examined the field notes to identify positive aspects of the E-MAT tremor software as well as areas for improvements noted by the occupational therapist and the patient.

### Case Study

The Occupational Therapist.

The participating occupational therapist began her career as an occupational therapy assistant working in the school system. After working for several years as an occupational therapy assistant, she went back to college and earned a Bachelor's in Science in occupational therapy. She has been practicing as an occupational therapist for ten years, with experience in acute care, inpatient rehabilitation, long-term care, and outpatient therapy. She currently works in an outpatient neurological rehabilitation clinic, and she has been practicing at this specialized facility for four and a half years. The occupational therapist has experience working with individuals who have sustained cerebrovascular accidents, spinal cord injuries, traumatic brain injuries, and those with degenerative neurological conditions. At any given point in time, approximately 10-15% of her caseload includes individuals who experience tremor. She typically provides compensatory interventions for those who experience tremor to increase their participation in daily activities.

The Patient.

The participating patient is a 37 year old African American female with a history of drug abuse. She was hospitalized one year ago for bilateral weakness secondary to a cerebrovascular accident. Since the hospitalization, the patient has been experiencing tremor in her left upper extremity. The patient is right-side dominant. However, the patient recently sought out outpatient therapy services to address the tremor and upper extremity weakness. Upon her initial evaluation, the patient identified cooking and dressing as areas in which tremor had the greatest impact on her level of functioning. Therapy goals included increasing left digit and hand strength and coordination to increase functional performance, as well as utilizing compensatory strategies to manage tremor during daily activities. Specific compensatory strategies that have been used in therapy to manage the tremor include splinting for stability as well as using weights during functional activities. For example, the patient wears a custom fabricated wrist cock-up splint during the majority of the day to provide stability to manage the tremor in her left hand during typical daily activities.

### Occupational Therapist Perspective

The occupational therapist felt that the attachment method used to secure the MotionNode to the patient's skin was helpful in obtaining readings relative to the frequency and amplitude of the tremor. The therapist explained that she did not notice the MotionNode move throughout the entire session, which she felt was a positive aspect of the equipment design. The therapist also described the E-MAT tremor software as a tool that could be used with anyone who experiences tremor; use of the tool is not confined to certain diagnoses. The therapist pointed out that she was not aware of, nor did she use any other type of objective measurement for tremor in the clinic. She explained that the measurements made by the E-MAT seemed accurate when compared to her observations. For example, the therapist stated, “It picks up that the tremor slows down whenever she talks and that the tremor goes faster whenever she focuses on the task or tries to rush, which is what I see also.”

Another aspect of the E-MAT software identified by the therapist was the manner in which data was reported. The therapist found it helpful that the software reported a specific number to describe the frequency and the amplitude of the tremor after taking a measurement. She stated, “This gives more concrete information than just my observation.” The occupational therapist typically does not use any objective observations or measures of tremor in the clinic. However, when comparing the measurements made by the E-MAT to those made by the UPDRS throughout therapy, the occupational therapist explained that she preferred the E-MAT to the UPDRS because the ratings made by the E-MAT were more concrete and consistent. She explained that, “The rating scale is a matter of opinion; [there is] no number, and [ratings] can vary at times.” She explained that the E-MAT was better capable of capturing the variability in tremor presentation than was the UPDRS, as the patient's tremor could be any/all of the ratings on the scale throughout the course of the therapy session. The therapist stated, “The E-MAT was more consistent in ratings, [and it] gave a bigger picture of how [the patient] was doing.”

Accordingly, the E-MAT could serve in a typical therapy session. For example, the E-MAT software would be beneficial in planning therapy sessions, selecting treatments, writing goals, and measuring outcomes in therapy. The E-MAT would be helpful for a treating occupational therapist to literally see which compensatory strategies were working best for managing tremor in a specific patient. Such information could then be incorporated to choose the best treatment options for the specific patient. For example, in the current study, the patient's tremor decreased whenever she wore the wrist cock-up splint. Therefore, the therapist had concrete evidence that the splint was an effective intervention strategy. The therapist stated that the numbers reported by the E-MAT would be beneficial for patients. She stated, “It is good feedback for the therapist and patient to see the numbers; [the numbers] make therapy more concrete.” The therapist explained that the patient could incorporate the feedback provided by the E-MAT into choosing to use specific interventions at home. The therapist described that the E-MAT software would be beneficial in demonstrating progress made in therapy, which may be helpful in obtaining insurance reimbursement for therapy services. The therapist stated, “You have the numbers in black and white; it's not my guess.”

Other Potential Uses of E-MAT Software.

The tremor application of the E-MAT software can be useful for other healthcare professionals. For example, neurologists could use the software to aid in diagnosing the underlying cause of tremor. Neurologists can also use the tremor software to determine the effectiveness of pharmacological management of tremor and to adjust medication regimens accordingly. Neurologist and surgeons can also use the E-MAT tremor application to determine the effectiveness of deep brain stimulation, which is a technique used to remediate tremor. Occupational and physical therapists can use the current software to measure management of tremor in the lower extremities, as well as in the upper extremities.

### Patient Perspective

Positive Aspects of E-MAT Software.

The patient identified several positive aspects of the E-MAT design. The patient stated several times that the MotionNode was “not uncomfortable.” The patient stated that the readings made by the E-MAT educated her about her situation. She said, “It helped me see how fast and slow [the tremor was moving] with certain activities, and how the tremor changed with the different activities.” The measurements made by the E-MAT also provided the patient with insights about strategies that were effective in managing her tremor. For example, the patient stated, “I didn't realize it,” whenever the therapist pointed out that the speed of her tremor decreased whenever she engaged in conversations with the therapist and that the speed of her tremor increased whenever she focused on an activity and whenever she tried to rush through a task to complete it. Overall, the patient stated that she liked the E-MAT because it showed her what helped manage her tremor. For example, the patient realized that she was able to perform therapy activities with less tremor interference when wearing her splint and when taking her time to complete activities.

Clinical Utility of E-MAT.

The patient stated that she believed that the E-MAT could be helpful for both the patient and the occupational therapist during therapy. The patient said, “I never seen nothing like that [the E-MAT] before, but it does show me what is going on.” The patient explained that the E-MAT allowed her to see which activities were difficult for her. She also explained that the feedback provided by the E-MAT was helpful when choosing which strategies to use to manage her tremor. For example, the patient stated that she did not notice that her tremor worsened whenever she focused or rushed through an activity. She said, “That is a strategy that I can use . . . not focusing because I can get frustrated.” The patient explained that the therapist could use the E-MAT in therapy to choose interventions that had the most positive impact on managing the tremor.

| | Occupational Therapist | Patient |
|---|---|---|
| Category | Perspective | Perspective |
| Positive aspects | Secure attachment method | Comfortable |
| of E-MAT | Useful for many diagnoses | Educate about own |
| software | Accurate measurements | situation |
| | compared to observation | Insights on |
| | Reported concrete numbers | managing tremor |
| | Concrete and consistent | |
| | ratings | |
| Clinical | Planning sessions | Determination of most |
| utility of | Selecting treatments | difficult activities |
| E-MAT | Writing goals | Personal choice of |
| | Measuring outcomes | management strategies |
| | Obtaining insurance | Therapist choice of |
| | reimbursement | interventions and activities |
| | Patient can incorporate | |
| | feedback at home | |

### Application to Occupational Therapy

Both the occupational therapist and the patient believed that the E-MAT provided a worthwhile clinical function in measuring the severity of tremor. The participating occupational therapist recognized a lack of objective measurements of tremor available in clinical practice. Her report is consistent with current literature, which has also identified a lack of objective measures of tremor in clinical settings (Bonato, 2005; Caligiuri & Tripp, 2004; DeGoede et al., 2012; Politis et al., 2010; Rigas et al., 2012; Salarian et al., 2007). Due to the lack of objective measures of tremor, the participating occupational therapist reported that she primarily relied on clinical observation during therapy to determine the effectiveness of intervention strategies. This too is consistent with the literature, which reports that clinicians primarily rely on observation to determine the severity of tremor in every day practice (Alty & Kempster, 2011; Bajaj et al., 2010). Because there is no way to quantify observation in a manner that it consistent across raters (Alty & Kempster, 2011; Bajaj et al., 2010), it is important that objective biomechanical assessments of tremor are developed. While the majority of available literature has focused on creating biomechanical assessment tools to aid in the diagnosis and pharmacological management of tremor (Caligiuri & Tripp, 2004; Rigas et al., 2012; Salarian et al., 2007), the present test of the tremor software of the E-MAT system applies an objective measure of tremor to a rehabilitation setting to measure the effectiveness of compensatory interventions on managing tremor, as this is an important area that has been neglected in previous research.

Results indicate that both the occupational therapist and the patient value the ability of the E-MAT to provide objective measures of tremor frequency and amplitude in a clinical setting. The occupational therapist recognized the place of the E-MAT system in planning intervention sessions, selecting the best treatment options for patients, writing measurable goals, measuring therapy outcomes, and potentially obtaining insurance reimbursement for therapy services. The patient recognized the place of the E-MAT system in therapy as assisting in identifying the most effective intervention strategies. The therapist and patient identified that the information gained from the E-MAT relative to which strategies were most effective in measuring tremor could be utilized by the patient outside of the therapy setting. For example, in the current study, the patient realized that taking her time to complete a task decreased the severity of her tremor. The patient could use this strategy to complete tasks in the therapy environment as well as in her home environment.

Based on the findings of the present study, it is clear that there is a place for the E-MAT in measuring the effectiveness of compensatory therapy on tremor management in a clinical setting. The E-MAT system offers a low cost option for objectively measuring the effectiveness of therapeutic interventions on managing tremor severity. Use of the E-MAT in a clinical setting can aid in planning intervention sessions, selecting the best treatment options for patients, writing measurable goals, measuring therapy outcomes, and potentially obtaining insurance reimbursement for therapy services.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An electronic motion assessment tool, comprising:
a plurality of motion nodes, wherein each motion node comprises an accelerometer, gyroscope, and magnetometer; and
a means for attaching the motion nodes to at least two joint regions of a subject.

2. The electronic motion assessment tool of claim 1, further comprising an Electronic-Movement Tracker (E-TRK).

3. The electronic motion assessment tool of claim 2, further comprising an Electronic-Movement Logger (E-LOG).

4. The electronic motion assessment tool of claim 3, further comprising a wearable computing device for logging joint movement of the subject.

5. The electronic motion assessment tool of claim 2, further comprising a motion node placement guide.

6. The electronic motion assessment tool of claim 2, wherein the plurality of motion nodes record range of motion data.

7. The electronic motion assessment tool of claim 2, further comprising means for graphing rotation, flexion, and abduction motions of a targeted joint in real time.

8. The electronic motion assessment tool of claim 6, further comprising means for generating rhythmic sound cues for assistance in motion emulation.

9. The electronic motion assessment tool of claim 2, further comprising means for measuring frequency, amplitude and smoothness of movement.

10. The electronic motion assessment tool of claim 9, wherein the measured movement is used in the assessment of tremor.

11. The electronic motion assessment tool of claim 10, wherein the measurement is on a graduated scale.

12. The electronic motion assessment tool of claim 2, further comprising means for generating audio cues for patients with visual impairments.

13. The electronic motion assessment tool of claim 2, further comprising means for generating hard copy reports of session results.

14. The electronic motion assessment tool of claim 2, further comprising means for generating real time range of motion displays.

15. The electronic motion assessment tool of claim 6, further comprising means to emulate a previously recorded motion.

16. The electronic motion assessment tool of claim 2, further comprising means for interactive spatial visualization and emulation exercise.

17. The electronic motion assessment tool of claim 16, wherein the visualization and emulation exercise further comprises a ball image.

18. The electronic motion assessment tool of claim 17, wherein the ball image translates up/down, left/right and rotates in response to joint rotations flexio/extension, ab/adduction and internal/external rotation, respectively.

19. The electronic motion assessment tool of claim 15, further comprising means to assess movement via a comparison of emulated motion to target with respect to deviation from the target rotations.

20. A method of assessing tremor of a body part of a subject, comprising:
positioning at least one motion node on or adjacent to the body part of the subject to be assessed, wherein the at least one motion node measures frequency, amplitude and smoothness of movement on a graduated scale; and
assessing tremor of the body part based on the measured values.
